(19) United States
(12) Patent Application Publication
(10) Pub. No.: US 2019/0038542 A1
(43) Pub. Date: Feb. 7, 2019

(54) MOISTURIZING PERSONAL CARE COMPOSITIONS COMPRISING MONODISPERSE PHYTOGLYCOGEN NANOPARTICLES AND A FURTHER POLYSACCHARIDE

(71) Applicant: MIREXUS BIOTECHNOLOGIES INC., Guelph (CA)

(72) Inventors: ANTON KORENEVSKI, GUELPH (CA); CARLEY MIKI, Guelph (CA); MARTIN KURYLOWICZ, Guelph (CA); ALISON CRUMBLEHULME, Burlington (CA)

(21) Appl. No.: 16/075,405

(22) PCT Filed: Aug. 26, 2016
(86) PCT No.: PCT/CA2016/000220

§ 371 Date: Aug. 3, 2018

Related U.S. Application Data
(60) Provisional application No. 62/292,604, filed on Feb. 8, 2016.

(51) Int. Cl. A61K 8/73; A61K 8/9794; A61K 8/34; A61Q 19/00

(52) U.S. Cl. CPC A61K 8/735; A61K 8/736; A61K 8/733; A61K 8/9794; A61K 8/345; A61K 8/046; A61K 2800/10; A61K 2800/52; A61K 2800/26; A61K 2800/413; A61Q 19/00

(57) ABSTRACT

Compositions comprising monodisperse phytoglycogen nanoparticles and a further moisturizing polysaccharide are disclosed as exhibiting a synergistically enhanced ability to moisturize the skin. Preferably, the phytoglycogen is isolated from plant matter such as sweet corn kernels. The moisturizing polysaccharide may be a glycosaminoglycan, a chitosan, an alginate, or a beta glucan. In preferred embodiments, the polysaccharide is hyaluronic acid. The compositions may optionally further comprise various carriers, fillers, and active agents.

This application claims priority from U.S. Patent Application No. 62/292,604.

### TECHNICAL FIELD

This invention relates to personal care compositions.

### BACKGROUND OF THE ART

Phytoglycogen is a plant-based energy storage material. It is a polysaccharide comprised of 1,4-glucan chains, highly branched via α-1,6-glucosidic linkages with a molecular weight of 10⁶-10⁸ Daltons. The most prominent sources of phytoglycogen are kernels of sweet corn, as well as specific varieties of rice, barley, and sorghum.

Japanese patent application JP1999000044901 proposes the use of phytoglycogen as an additive for hair formulations that imparts improved combing properties and shiny appearance to hair.

Hyaluronic acid (sometimes called hyaluronan or hyaluronate) is an anionic, nonsulfated glycosaminoglycan distributed throughout connective, epithelial and neural tissues. Hyaluronic acid is commercially available and has been used in medical and cosmetic applications.

There is a growing need for incorporation of natural, non-toxic and biodegradable materials in personal care products to replace petroleum-based chemicals.

### BRIEF SUMMARY

In one embodiment, there is provided a personal care composition comprising a monodisperse phytoglycogen nanoparticle component and a moisturizing polysaccharide component. In one embodiment, the moisturizing polysaccharide component comprises at least one of a glycosaminoglycan, a chitosan, an alginate or a beta glucan or a similar or derivative polysaccharide.

In one embodiment, the moisturizing polysaccharide component comprises a glycosaminoglycan or a derivative thereof, in one embodiment hyaluronic acid.

In one embodiment, the moisturizing polysaccharide component comprises a chitosan, an alginate and/or a beta glucan.

In one embodiment, the composition is a water-based formulation. In one embodiment, the composition is an alcohol-based formulation. The alcohol can suitably be ethyl alcohol, propyl alcohol, isopropyl alcohol, ethylene glycol, propylene glycol, butylene glycol, dipropylene glycol, ethoxydiglycol, or glycerol or a combination thereof.

In one embodiment, the phytoglycogen nanoparticle component has a polydispersity index of less than about 0.3, 0.2 or 0.1 as measured by dynamic light scattering.

In one embodiment, at least about 80% by dry weight of the phytoglycogen nanoparticle component is monodisperse phytoglycogen nanoparticles having an average particle diameter of between about 30 nm and about 150 nm.

In one embodiment, about 90% by dry weight of the phytoglycogen nanoparticle component is monodisperse phytoglycogen nanoparticles having an average particle diameter of between about 30 nm and about 150 nm.

In one embodiment, about 90% by dry weight of the phytoglycogen nanoparticle component is monodisperse phytoglycogen nanoparticles having an average particle size of between about 60 nm and about 110 nm.

In one embodiment, the monodisperse phytoglycogen nanoparticles are chemically modified. The monodisperse phytoglycogen nanoparticles may be modified by chemical functionalization of at least one of its hydroxyl groups with a carbonyl group, an amine group, a thiol group, a carboxylic group, or a hydrocarbyl, the hydrocarbyl group may be an alkyl, vinyl or allyl group. In one embodiment, the monodisperse phytoglycogen nanoparticles are modified with octenyl succinic acid.

In one embodiment, the phytoglycogen nanoparticle component and the moisturizing polysaccharide component are present in a concentration of up to about 25% w/w, in one embodiment, between about 0.05 and about 5% w/w.

In one embodiment, the phytoglycogen nanoparticle component and the moisturizing polysaccharide component are present in substantially equal amounts.

The personal care formulation may be a lotion, a gel, a mask, a sunscreen, a sanitizer, a shampoo, a conditioner, a deodorant, an antiperspirant or a cosmetic.

In one embodiment, the formulation further comprises a natural gum.

In one embodiment, the composition further includes at least one small molecule, polymer, biopolymer, colloidal particle or an oil.

In one embodiment, the composition further includes a cosmetically-acceptable carrier, which may include one or more ingredients selected from absorbents, anti-acne agents, anti-caking agents, anti-cellulite agents, anti-foaming agents, anti-fungal agents, anti-inflammatory agents, anti-microbial agents, antioxidants, antiperspirant/deodorant agents, anti-viral agents, anti-wrinkle agents, artificial tanning agents, astringents, binders, buffering agents, bulking agents and fillers, chelating agents, colouring agents and dyes, emollients, emulsifiers, enzymes, essential oils, exfoliating agents, film formers, flavors, foaming agents, fragrances and perfumes, humectants, hydrating agents, hydrocolloids, light diffusers, lightening agents, opacifiers, optical brighteners and modifiers, particulates, pH adjusters, preservatives, sequestering agents, skin conditioners and moisturizers, skin feel modifiers, skin protectants, skin soothing and healing agents, solvents, stabilisers, sunscreen actives, topical anesthetics, viscosity modifiers, vitamins, and combinations thereof.

The composition may be in the form of a powder, a liquid or a gel. In one embodiment, the composition is a spray on personal care composition, which may be a spray on cosmetic, sunscreen, deodorant, antiperspirant, aftershave or hand sanitizer or a hairspray.

### BRIEF DESCRIPTION OF THE DRAWINGS

FIG. 1 shows the viscosity of monodisperse phytoglycogen nanoparticles in water at different concentrations.

FIG. 2 shows the shear rate dependence on viscosity of a dispersion of monodisperse phytoglycogen nanoparticles in water.

FIG. 3 shows the flow behavior of a dispersion of monodisperse phytoglycogen nanoparticles at 19% (w/w) is independent of shear rate.

FIG. 4 shows peak hold flow tests of a 0.5% (w/w) konjac gum solution with and without 0.5% monodisperse phytoglycogen.

FIG. 5A shows viscosity values in stepped flow loops of compositions containing 0.5% konjac gum without phytoglycogen nanoparticles of the present invention.

FIG. 5B shows viscosity values in stepped flow loops of the compositions containing 0.5% konjac gum with 0.5% phytoglycogen.

FIG. 6 compares viscosity values in a temperature sweep cycle between 0 and 50° C., with increasing temperature in the first part of the cycle and decreasing temperature in the second part of the cycle for cream base without phytoglycogen nanoparticles (squares) and with phytoglycogen nanoparticles (circles).

FIG. 7 shows photostability of aminocinnamate. (diamonds) and a phytoglycogen-ethyl-4-aminocinnamate conjugate (squares).

FIG. 8 shows relative water retention of a dried monodisperse phytoglycogen nanoparticle composition, glycerol, PEG 400 and hyaluronic acid.

FIG. 9 shows the viscosity values of dispersions of monodisperse phytoglycogen nanoparticles show no significant dependence on the ionic strength.

FIG. 10 shows the percentage change in skin hydration at 1, 3, 5 and 7 hours post-application for base moisturizer and base moisturizer plus 0.1% monodisperse phytoglycogen nanoparticles; 0.1% hyaluronic acid; and 0.05% monodisperse phyhoglycogen nanoparticles plus 0.05% hyaluronic acid.

FIG. 11 shows the results of blind testing of otherwise equivalent moisturizers including hyaluronic acid or monodisperse phytoglycogen nanoparticles.

FIG. 12 shows scanning Electron Microscope (SEM) images of a cross-section of pure chitosan film (A) and a 35:65 blend of phytoglycogen:chitosan film (B).

FIG. 13 shows equal parts sodium hyaluronate and phytoglycogen film, cast from 50 mL 0.5% sodium hyaluronate, 0.5% phytoglycogen solution.

FIG. 14 shows equal parts sodium alginate and phytoglycogen film, cast from 50 mL 1% sodium hyaluronate, 1% phytoglycogen solution.

FIG. 15 shows the zero-shear viscosity (A) and yield stress (B) of 2.2% hyaluronic acid solutions in PBS, with varying phytoglycogen nanoparticle content.

FIG. 16 shows (A) shear-thinning behavior of 1% hyaluronic acid solutions containing various concentrations of phytoglycogen; (B) viscosity of 1% hyaluronic acid solutions containing various concentration of phytoglycogen at 100 s-1 shear rate; (C) zero-shear viscosity of 1% hyaluronic acid solutions containing various phytoglycogen concentrations.

FIG. 17 shows dehydration of fully hydrated powders containing varying quantities of hyaluronic acid and phytoglycogen nanoparticles. Powders with a higher phytoglycogen nanoparticle content retained the most water over a 24 hr period.

FIG. 18 shows the results of a sensory evaluation of creams containing either 0.1% hyaluronic acid or 0.05% hyaluronic acid+0.05% phytoglycogen nanoparticles.

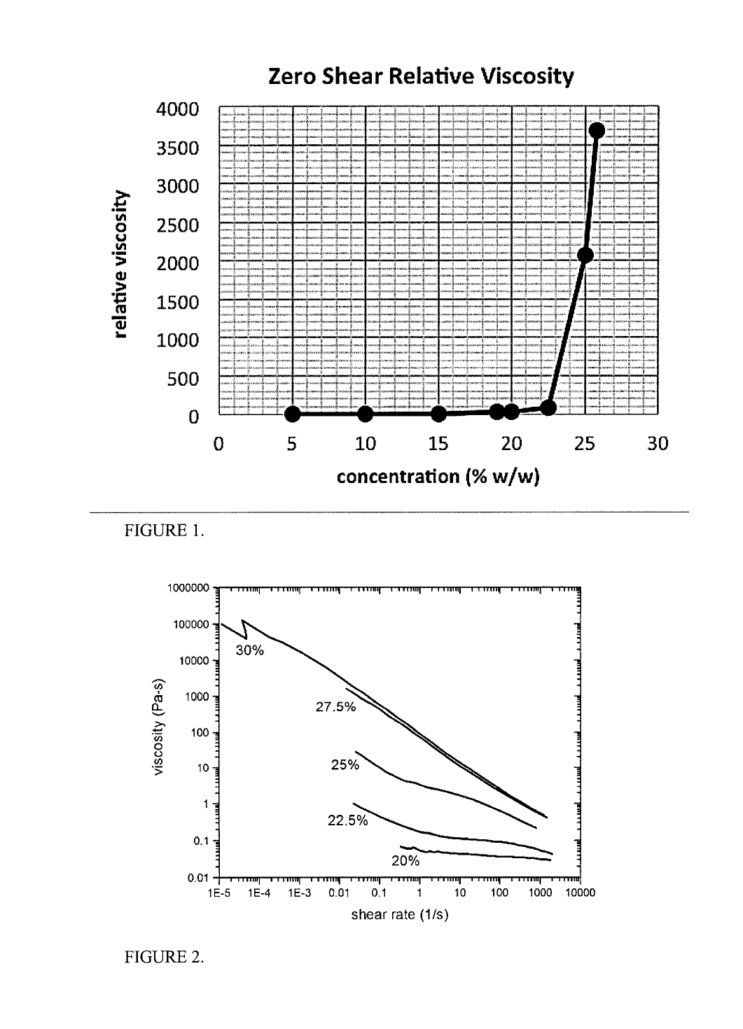

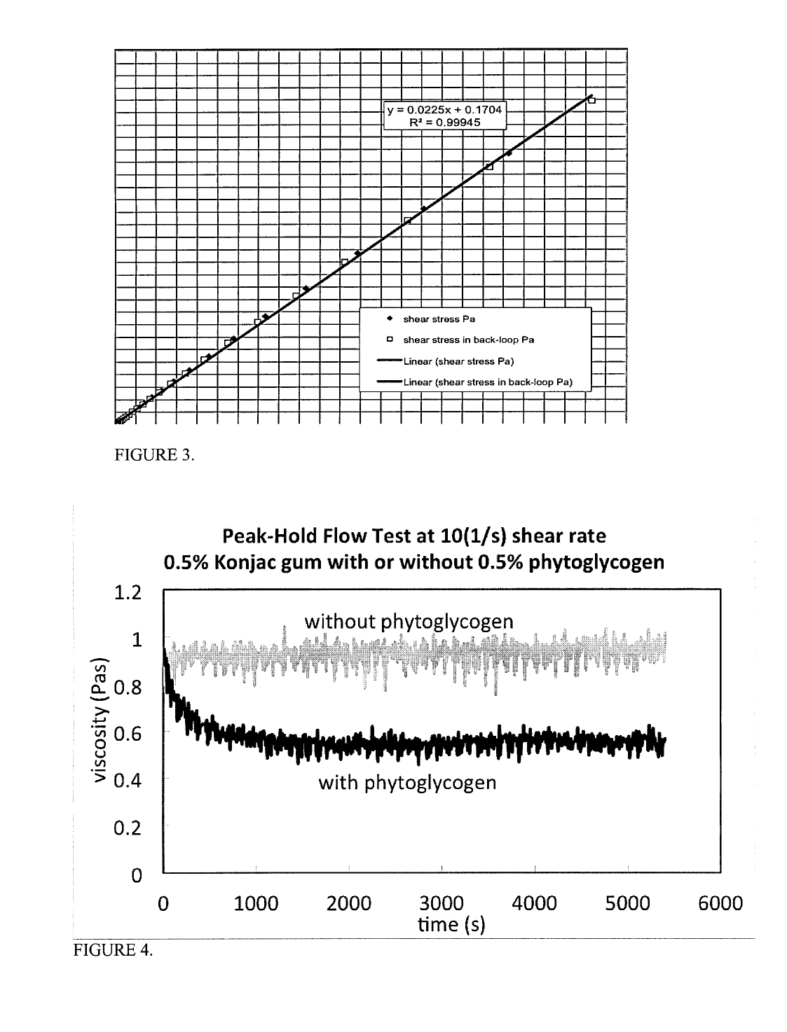

### DETAILED DESCRIPTION

In one embodiment, there is described a personal care product comprising phytoglycogen, preferably monodisperse nanoparticles of phytoglycogen, and at least one other moisturizing polysaccharide. Reference may be made below to a “phytoglycogen component” or “phytoglycogen nanoparticle component” and a “moisturizing polysaccharide component”. The moisturizing polysaccharide component may be composed of a single polysaccharide or a combination of polysaccharides. In one embodiment, a moisturizing polysaccharide component comprises a polysaccharide that applied alone whether directly or in a suitable solvent or cosmetic base, as appropriate, moisturizes human skin. Moisturizers, including moisturizing polysaccharides are known, and may act by forming a thin film on the surface of the skin to prevent loss of moisture and/or attracting water vapor from the air to moisturize the skin. In one embodiment, the moisturizing polysaccharide is a naturally-derived, non-storage non-cellulosic polysaccharide; while in one embodiment, the moisturizing polysaccharide component is not specifically limited, in various embodiments, the moisturizing polysaccharide component comprises, consists of or consists essentially of one or more of a glycosaminoglycan (in one embodiment, hyaluronic acid), chitosan, an alginate, an a beta-glucan or a derivative thereof or a similar polymer.

In one embodiment, the polysaccharide(s) of the polysaccharide component have a molecular weight of up to 2000 kDa, in one embodiment, between about 5 kDa and 2000 kDa, in one embodiment between about 500 kDa and 1500 kDa.

In one embodiment, the at least one other polysaccharide is a glycosaminoglycan. In one embodiment, the glycosaminoglycan is hyaluronic acid or a derivative thereof. In one embodiment, hyaluronic acid. In other embodiments, the moisturizing polysaccharide component may include, in the alternative, or in addition, a chitosan, an alginate and/or a beta glucan or a derivative thereof.

In one embodiment, the moisturizing polysaccharide is not a natural gum. In one embodiment, the moisturizing polysaccharide is selected from hyaluronic acid, chitosan and alginate and derivatives thereof or mixtures thereof.

While in one embodiment, not so limited, in one embodiment, the polysaccharide ingredients of a personal care composition as provided herein are limited or limited essentially to the phytoglycogen component and the moisturizing polysaccharide component. In one embodiment, the polysaccharide ingredients of a personal care composition as provided herein are limited to the phytoglycogen component and the moisturizing polysaccharide component and one or more natural gums.

In one embodiment, the moisturizing polysaccharide component used in formulating the personal care composition is a powder or particulate.

As detailed in the Examples, the present inventors have determined that monodisperse phytoglycogen nanoparticles can impart personal care formulations with beneficial properties, however, the inventors have further determined that a personal care formulation that includes both a monodisperse phytoglycogen nanoparticle component and another moisturizing polysaccharide component e.g. hyaluronic acid displays hydrating effect superior to an equivalent amount of either component alone.

Phytoglycogen is composed of molecules of α-D glucose chains having an average chain length of 11-12, with 1→4 linkage and branching point occurring at 1→6 and with a branching degree of about 6% to about 13%.

In one embodiment phytoglycogen includes both phytoglycogen derived from natural sources and synthetic phytoglycogen. As used herein “synthetic phytoglycogen” includes glycogen-like products prepared using enzymatic processes on plant-derived material e.g. starch.

While in one disclosed embodiment, phytoglycogen used in any novel compositions and methods described herein can be obtained using any known method or be obtained from a commercial source, the commercial products and yields of most methods are highly polydisperse products that include both phytoglycogen particles, as well as other products and degradation products of phytoglycogen, which may render them less effective in the compositions and methods described herein. In a preferred embodiment, monodisperse phytoglycogen nanoparticles are used. In one embodiment, the monodisperse phytoglycogen nanoparticles are PhytoSpherix™ manufactured by Mirexus Biotechnologies, Inc. The monodisperse and particulate nature of these nanoparticles are associated with properties that render them highly suitable for use in the described personal care compositions and, in particular as shown in the Examples evidence a synergistic effect in personal care compositions comprising another moisturizing polysaccharide component, which may in one embodiment, be a glycosaminoglycan and, in particular, hyaluronic acid.

In one embodiment, phytoglycogen refers to monodisperse nanoparticles of phytoglycogen manufactured according to methods disclosed herein.

In one embodiment, the personal care compositions may be water-based formulations, which may include, in particular, dispersions, including emulsions and suspensions, and solutions of one or more of small molecules, polymers, biopolymers, colloidal particles and oils.

The personal care compositions may be alcohol-based formulations, which may include in particular, dispersions, including emulsions and suspensions, and solutions of one or more of small molecules, polymers, biopolymers, colloidal particles and oils in one or more alcohols. Alcohols may be selected from, but are not limited to, ethyl alcohol, propyl alcohol, isopropyl alcohol, ethylene glycol, propylene glycol, butylene glycol, dipropylene glycol, ethoxydiglycol, glycerol and mixtures thereof.

In various embodiments, the phytoglycogen nanoparticle component as described herein may suitably be used in the personal care compositions in a concentration of up to about 25% w/w, about 20% w/w, about 15% w/w, about 10% w/w, about 5% w/w, about 1% w/w and between about 0.05 and 0.5%.

In applications where a high viscosity is desirable, the phytoglycogen nanoparticle component may be used in formulations in concentrations above about 25% w/w. In applications where a gel or semi-solid is desirable, concentrations up to about 35% w/w can be used.

In various embodiments, the moisturizing polysaccharide component as described herein may suitably be used in the personal care compositions in a concentration of up to about 25% w/w, about 20% w/w, about 15% w/w, about 10% w/w, about 5% w/w, about 1% w/w and between about 0.05 and 0.5%.

The phytoglycogen nanoparticle component and the moisturizing polysaccharide component may be present in equal or about equal amounts or may be present in differing amounts. In one embodiment, both components are present in an amount of less than about 5% w/w and in an amount ≥0.05% w/w.

The phytoglycogen nanoparticle component is non-toxic, has no known allergenicity, and can be degraded by glycogenolytic enzymes (e.g. amylases and phosphorylases) of the human body. The products of enzymatic degradation are non-toxic molecules of glucose.

The phytoglycogen component is compatible with most personal care formulation ingredients such as emulsifiers, surfactants, thickeners, preservatives, and physical and chemical sunscreen active ingredients.

As detailed below, the phytoglycogen nanoparticle component is photostable and is also stable over a wide range of pH, electrolytes, e.g. salt concentrations.

Methods of manufacturing monodisperse compositions of phytoglycogen are disclosed in the International patent application entitled “Phytoglycogen Nanoparticles and Methods of Manufacture Thereof”, published under the international application publication no WO2014/172786 and the disclosure of which is incorporated by reference in its entirety. In one embodiment, the described methods of producing monodisperse phytoglycogen nanoparticles include: a. immersing disintegrated phytoglycogen-containing plant material in water at a temperature between about 0 and about 50° C.; b. subjecting the product of step (a.) to a solid-liquid separation to obtain an aqueous extract; c. passing the aqueous extract of step (b.) through a microfiltration material having a maximum average pore size of between about 0.05 μm and about 0.15 μm; and d. subjecting the filtrate from step c. to ultrafiltration to remove impurities having a molecular weight of less than about 300 kDa, in one embodiment, less than about 500 kDa, to obtain an aqueous composition comprising monodisperse phytoglycogen nanoparticles. In one embodiment of the method, the phytoglycogen-containing plant material is a cereal selected from corn, rice, barley, sorghum or a mixture thereof. In one embodiment, step c. comprises passing the aqueous extract of step (b.) through (c.1) a first microfiltration material having a maximum average pore size between about 10 μm and about 40 μm; (c.2) a second microfiltration material having a maximum average pore size between about 0.5 μm and about 2.0 μm, and (c.3) a third microfiltration material having a maximum average pore size between about 0.05 and 0.15 μm. The method can further include a step (e.) of subjecting the aqueous composition comprising monodisperse phytoglycogen nanoparticles to enzymatic treatment using amylosucrose, glycosyltransferase, branching enzymes or any combination thereof. The method avoids the use of chemical, enzymatic or thermo treatments that degrade the phytoglycogen material. The aqueous composition can further be dried.

The polydispersity index (PDI) of a composition of nanoparticles can be determined by the dynamic light scattering (DLS) technique and, in this embodiment, PDI is determined as the square of the ratio of standard deviation to mean diameter (PDI=(σ/d)². PDI can also be expressed through the distribution of the molecular weight of polymer and, in this embodiment, is defined as the ration of M_{w} to Mₙ, where M_{w} is the weight-average molar mass and Mₙ is the number-average molar mass (hereafter this PDI measurement is referred to as PDI*). In the first case, a monodisperse material would have a PDI of zero (0.0) and in the second case the PDI* would be 1.0.

In one embodiment, the phytoglycogen nanoparticle component comprises, consists essentially of, or consists of monodisperse phytoglycogen nanoparticles, having, in various embodiments, a PDI of less than about 0.3, less than about 0.2, less than about 0.15, less than about 0.10, or less than 0.05 as measured by dynamic light scattering. The phytoglycogen nanoparticle component may comprise, consist essentially of, or consist of monodisperse phytoglycogen nanoparticles having a PDI* of less than about 1.3, less than about 1.2, less than about 1.15, less than about 1.10, or less than 1.05 as measured by SEC MALS

In one embodiment, the phytoglycogen nanoparticle component comprises, consists essentially of, or consists of monodisperse phytoglycogen nanoparticles having an average particle diameter of between about 30 nm and about 150 nm. In one embodiment, the phytoglycogen nanoparticle component comprises, consists essentially of, or consists of monodisperse phytoglycogen nanoparticles having an average particle diameter of about 60 nm to about 110 nm. In one embodiment, the phytoglycogen nanoparticles may have an average molecular weight of between about 4500 and 22000 kDa.

Due to the source of raw material used for manufacturing, monodisperse phytoglycogen nanoparticles and the origin of polysaccharides identified herein, personal care compositions as described herein may be identified as natural or organic formulations.

The methods of producing phytoglycogen nanoparticles as detailed in Example 1 and as taught in the international patent application entitled “Phytoglycogen Nanoparticles and Methods of Manufacture Thereof” are amenable to preparation under food grade conditions.

In one embodiment, the phytoglycogen is modified. Functionalization can be carried out on the surface of the nanoparticle, or on both the surface and the interior of the particle, but the structure of the phytoglycogen molecule as a single branched homopolymer is maintained. In one embodiment, the functionalization is carried out on the surface of the nanoparticle.

As will be apparent to persons of skill in the art, the chemical modifications should be non-irritating when in contact with human skin.

In some embodiments, the chemical character of phytoglycogen nanoparticles produced according to methods described above may be changed from their hydrophilic, slightly negatively charged native state to be positively and/or negatively charged, or to be partially or highly hydrophobic. Chemical processing of polysaccharides is well known in the art. See for example J. F Robyt, Essentials of Carbohydrate Chemistry, Springer, 1998; and M. Smith, and J. March, March's Advanced Organic Chemistry: Reactions, Mechanisms, and Structure Advanced Organic Chemistry, Wiley, 2007.

The nanoparticles can be either directly functionalized or indirectly, where one or more intermediate linkers or spacers can be used. The nanoparticles can be subjected to one or more than one functionalization steps including two or more, three or more, or four or more functionalization steps.

Various derivatives can be produced by chemical functionalization of hydroxyl groups of phytoglycogen. Such functional groups include, but are not limited to, nucleophilic and electrophilic groups, and acidic and basic groups, e.g., carbonyl groups, amine groups, thiol groups, carboxylic groups, and hydrocarbyl groups such as alkyl, vinyl and allyl groups. Amino groups can be primary, secondary, tertiary, or quaternary amino groups.

In one embodiment, the phytoglycogen nanoparticles are modified using various derivatives of succinic acid to increase their hydrophobicity. In one embodiment, phytoglycogen is modified using octenyl succinic acid (OSA), resulting in phytoglycogen nanoparticles with partially hydrophobic functionality, with the degree of substitution between 0.1 and 0.4.

In some embodiments, functionalized nanoparticles can be further conjugated with various desired molecules, which are of interest for a variety of applications, such as biomolecules, small molecules, therapeutic agents, micro- and nanoparticles, pharmaceutically active moieties, macromolecules, diagnostic labels, chelating agents, dispersants, charge modifying agents, viscosity modifying agents, surfactants, coagulation agents and flocculants, as well as various combinations of these chemical compounds.

Known methods for polysaccharide functionalization or derivatization can be used. For example, one approach is the introduction of carbonyl groups, by selective oxidation of glucose hydroxyl groups at positions of C-2, C-3, C-4 and/or C-6. There is a wide spectrum of oxidative agents which can be used such as periodate (e.g., potassium periodate), bromine, dimethyl sulfoxide/acetic anhydride (DMSO/Ac₂O) [e.g., U.S. Pat. No. 4,683,298], Dess-Martin periodinane, etc.

The nanoparticles described herein when functionalized with carbonyl groups are readily reactive with compounds bearing primary or secondary amine groups. This results in imine formation which can be further reduced to amine with a reductive agent e.g., sodium borohydrate. Thus, the reduction step provides an amino-product that is more stable than the imine intermediate, and also converts unreacted carbonyls in hydroxyl groups. Elimination of carbonyls significantly reduces the possibility of non-specific interactions of derivatized nanoparticles with non-targeted molecules, e.g. plasma proteins.

The reaction between carbonyl- and amino-compounds and the reduction step can be conducted simultaneously in one vessel (with a suitable reducing agent introduced to the same reaction mixture). This reaction is known as direct reductive amination. Here, any reducing agent, which selectively reduces imines in the presence of carbonyl groups, e.g., sodium cyanoborohydrate, can be used.

For the preparation of amino-functionalized nanoparticles from carbonyl-functionalized nanoparticles, any ammonium salt or primary or secondary amine-containing compound can be used, e.g., ammonium acetate, ammonium chloride, hydrazine, ethylenediamine, or hexanediamine. This reaction can be conducted in water or in an aqueous polar organic solvent e.g., ethyl alcohol, DMSO, or dimethylformamide.

Reductive amination of the nanoparticles described herein can be also achieved by using the following two step process. The first step is allylation, i.e., converting hydroxyls into allyl-groups by reaction with allyl halogen in the presence of a reducing agent, e.g., sodium borohydrate. In the second step, the allyl-groups are reacted with a bifunctional aminothiol compound, e.g., aminoethanethiol.

Amino-functionalized nanoparticles are amenable to further modification. For example, amino groups are reactive to carbonyl compounds (aldehydes and ketones), carboxylic acids and their derivatives, (e.g., acyl chlorides, esters), succinimidyl esters, isothiocyanates, sulfonyl chlorides, etc.

In certain embodiments, the nanoparticles described herein are functionalized using the process of cyanylation. This process results in the formation of cyanate esters and imidocarbonates on polysaccharide hydroxyls. These groups react readily with primary amines under very mild conditions, forming covalent linkages. Cyanylation agents such as cyanogen bromide, and, preferably, 1-cyano-4-diethylamino-pyridinium (CDAP), can be used for functionalization of the nanoparticles.

Functionalized nanoparticles can be directly attached to a chemical compound bearing a functional group that is capable of binding to carbonyl- or amino-groups. However, for some applications it may be important to attach chemical compounds via a spacer or linker including for example a polymer spacer or a linker. These can be homo- or hetero-bifunctional linkers bearing functional groups which include, but are not limited to, amino, carbonyl, sulfhydryl, succimidyl, maleimidyl, and isocyanate e.g., diaminohexane, ethylene glycobis(sulfosuccimidylsuccinate) (sulfo-EGS), disulfosuccimidyl tartarate (sulfo-DST), dithiobis(sulfosuccimidylpropionate) (DTSSP), aminoethanethiol, and the like.

In certain embodiments, two or more different chemical compounds are used to produce multifunctional derivatives.

In one embodiment, phytoglycogen nanoparticles used in personal care compositions as described herein are not functionalized.

While in one embodiment, the moisturizing polysaccharide component is not specifically limited, in various embodiments, the moisturizing polysaccharide component comprises one or more of a glycosaminoglycan (e.g. hyaluronic acid), chitosan, an alginate, or a beta-glucan or derivative thereof.

Hyaluronic acid is a natural glycosaminoglycan that is a chief component of the extracellular matrix.

Polysaccharides, including chitosan and glycosaminoglycans and, in particular, hyaluronic acid suitable for use in personal care formulations are commercially available.

As mentioned above, methods of chemically processing polysaccharides are well-known [e.g. J. F Robyt, Essentials of Carbohydrate Chemistry, Springer, 1998; and M. Smith, and J. March, March's Advanced Organic Chemistry: Reactions, Mechanisms, and Structure Advanced Organic Chemistry, Wiley, 2007] and methods of preparing derivatives of moisturizing polysaccharides will be known to those of skill in the art. Suitable derivatives can include salts, esters, carboxylated, OSA-modified and cationized derivatives of these polysaccharides Known derivatives of chitosan include e.g. chitosan succinamide and N-succinyl-chitosan. Known derivatives of alginate include e.g. calcium alginate and propylene glycol alginate. Known derivatives of hyaluronic acid include sodium hyaluronate.

In one embodiment, a composition described herein may be provided in the form of dry powder or granulate, which may be used to produce a personal care composition by being combined with other components for example mixed or dispersed, in a formulation comprising e.g. water or alcohol or a cosmetic base.

Successfully incorporating certain polysaccharides e.g. hyaluronic acid into personal care formulations can pose challenges and combining these polysaccharides with phytoglycogen nanoparticles in solution prior to combining with other ingredients such as moisturizer base can facilitate the incorporation of the other polysaccharides into personal care formulations. Accordingly, in one embodiment there is provided a method of preparing a personal care composition containing a further moisturizing polysaccharide component e.g. glycosaminoglycan comprising combining the polysaccharide(s) with phytoglycogen nanoparticles in water and/or alcohol prior to adding these components to the personal care composition.

In one embodiment, the phytoglycogen nanoparticle component is provided in the form of a water solution having a concentration of up to 25% w/w. In one embodiment, the phytoglycogen nanoparticle component is provided in the form of a gel or semi-solid having a concentration of up to 35% w/w.

The present invention encompasses water-based and alcohol-based formulations that include the phytoglycogen nanoparticle component and at least one other polysaccharide component, e.g. a glycosaminoglycan and/or chitosan.

In one embodiment, formulations described further include a natural gum.

In one embodiment, the composition is a personal care product which may be, but is not limited to a skin lotion or gel, a mask, a sunscreen, a sanitizer (e.g. hand sanitizer), a shampoo or conditioner, a deodorant or antiperspirant or a cosmetic.

The personal care formulations as described herein may be coated onto or impregnated into a product, e.g. a wipe, towlette, patch or sponge.

Emulsion formulations containing alcohols dissolved into the water phase are often used in spray-on cosmetic products, e.g. sunscreens, antiperspirants, aftershaves, hand sanitizers, etc. This allows fast drying of such formulations on the skin surface.

In one embodiment, the formulation comprises an oil in alcohol dispersion. In one embodiment, the alcohol is not particularly restricted and suitable alcohols may be selected by those of skill in the art based on the use of the composition. Suitable alcohols include but are not limited to ethyl alcohol, propyl alcohol, isopropyl alcohol, ethylene glycol, propylene glycol, butylene glycol, dipropylene glycol, ethoxydiglycol, glycerol or a combination thereof.

Hydrophobically-modified phytoglycogen (e.g., OSA-modified phytoglycogen) allows the incorporation of oily compounds such as emollients, sunscreen agents, perfumes (fragrances), vitamins A, D and E, essential oils, etc. into aqueous alcohol-containing formulations, which significantly reduces or even eliminates the need for emulsifiers and solubilizing additives. The addition of OSA-modified phytoglycogen with degrees of substitution ranging from 0.05 to 0.3 results in stable dispersions in aqueous alcohol solutions with alcohol content up to 85%. The alcohol used in these solutions was selected from the following: ethyl alcohol, propyl alcohol, isopropyl alcohol, ethylene glycol, propylene glycol, butylene glycol, dipropylene glycol, ethoxydiglycol, etc., and combinations of these alcohols. Furthermore, the low viscosity of aqueous alcohol formulations containing hydrophobically-modified phytoglycogen makes it possible to use these formulations for spray-on applications for cosmetic, personal care and other products.

In one embodiment, the composition is a spray on personal care product. Spray on personal care products include, but are not limited to, spray on cosmetics, spray on sunscreens, hairsprays, spray on deodorants, spray on antiperspirants, spray on aftershaves and spray on hand sanitizers.

### Rheology

Monodisperse phytoglycogen nanoparticles can act as rheology modifiers in water-based and alcohol-based formulations and, in particular, solutions and dispersions of small molecules, polymers, biopolymers, colloidal particles or oils (e.g. emulsions). When used in suitable concentrations, the nanoparticle component modulates viscosity and visco-elastic properties.

Time dependent rheological behaviour can be useful in personal care applications where it can be highly desirable for a product to have a thick, high viscosity texture but to become liquid-like and easily pourable after shaking and then regaining its original properties shortly after it is allowed to rest.

When increasing the rate of shear stress applied to a material results in decreasing viscosity, the phenomenon is called shear thinning (the material which exhibits shear thinning behaviour is called pseudoplastic). Since it takes time to rebuild the inner structure of a pseudoplastic material when the mixing stops, by definition all shear thinning compositions are thixotropic. The time required for “re-thickening” is key in practical applications. The term of thixotropy is used when re-thickening takes a noticeable time by simple observation.

As detailed in the Examples, the phytoglycogen nanoparticle component can impart beneficial rheological properties, and, in particular, shear thinning, increasing re-thickening time in a pseudoplastic system or imparting thixotropic behavior in other viscoelastic systems.

### Stabilizer

The phytoglycogen component can confer increased phase stability, increased heat stability, and increased stability in storage to personal care formulations as illustrated in the Examples. Furthermore, this component acts as a photostabilizer for photolabile compounds, which are commonly used in various cosmetics. The component can provide improved emulsification and emulsion stabilization of oil in water emulsions, such as creams lotions etc.

For personal care formulations, stability (physical, chemical and photochemical) is a critical factor. Temperature has a significant effect on stability.

Viscosity hysteresis is common when cycling between heating and cooling phases during temperature cycling tests.

Monodisperse phytoglycogen nanoparticles may be used to improve emulsion stability on temperature cycling and to delay or prevent the “melting” (crossing between G′ and G″) of oil in water emulsions in the temperature range of 0-50° C.

In the preparation of emulsion-based personal care formulations (typically oil-in-water emulsions), it is necessary to heat both water and oil phases above 50° C. to create a well-mixed emulsion (base emulsion). However, sometimes it is desirable to introduce additional thermo-labile (heat sensitive), water-soluble components, e.g., preservatives, bioactives, fragrances, etc. into the formulations at lower temperatures, e.g. temperatures below 35° C. This can be achieved by cooling the base emulsion and mixing-in a third, water-based phase containing the heat sensitive ingredients. However, the viscosity of the end product will strongly depend on the mechanical technique used to mix the third phase into the base emulsion.

The presence of a phytoglycogen nanoparticle component as described herein can improve tolerance of the emulsions to mechanical stresses and enables faster, high-energy mixing techniques, e.g. homogenizers.

### UV Protection

The phytoglycogen nanoparticle component may also increase the SPF index of sun protection formulations whether based on physical (containing inorganic pigments) or chemical (containing UV-absorbing chemicals) sunscreen agents.

Modified (octenylsuccinic acid) and un-modified phytoglycogen nanoparticles have been incorporated into sunscreen formulations containing homosalate or titanium dioxide. Resulting sunscreen formulations demonstrated higher SPF values and improved photostability.

### Photostabilization of Formulations

Monodisperse phytoglycogen nanoparticles can act as a photostabilizer.

The photostabilizing effect of the phytoglycogen nanoparticle component is of particular utility in the case of organic sunscreen formulations. Numerous recent studies have demonstrated that many sunscreen actives suffer from inadequate photostability and rapidly lose their photoprotective ability upon ultraviolet irradiation. This results in marketed products that may not meet their labeled SPF index. Furthermore, photoinactivation of sunscreens may produce free radical intermediates and compounds that act as sensitizers and photoallergens.

To improve photostability and dispersibility, sunscreen actives can also be covalently conjugated to the phytoglycogen nanoparticles.

### Humectant

Monodisperse phytoglycogen nanoparticles can also improve water retention properties.

It has been shown that when phytoglycogen nanoparticles are fully hydrated ˜62% of their volume is filled with water and 1.64 g water/g phytoglycogen is part of the hydrated phytoglycogen structure. When the phytoglycogen nanoparticle component is allowed to come to equilibrium in a high moisture (98% RH) environment, it will absorb ˜50% of its own weight in water.

### Example 1. Extraction of Phytoglycogen from Sweet Corn Kernels

1 kg of frozen sweet corn kernels (75% moisture content) was mixed with 2 L of deionized water at 20° C. and was pulverized in a blender at 3000 rpm for 3 min. Mush was centrifuged at 12,000×g for 15 min at 4° C. The combined supernatant fraction was subjected to CFF using a membrane filter with 0.1 μm pore size. The filtrate was further purified by a batch diafiltration using membrane with MWCO of 500 kDa and at RT and diavolume of 6. (Diavolume is the ratio of total mQ water volume introduced to the operation during diafiltration to retentate volume.)

The retentate fraction was mixed with 2.5 volumes of 95% ethanol and centrifuged at 8,000×g for 10 min at 4° C. The retentate was mixed with 2.5 volumes of 95% ethanol and centrifuged at 8,000×g for 10 min at 4° C. The pellet containing phytoglycogen was dried in an oven at 50° C. for 24 h and then milled to 45 mesh. The weight of the dried phytoglycogen was 97 g.

According to DLS measurements, the phytoglycogen nanoparticles produced had particle size diameter of 83.0 nm and the polydispersity index of 0.081.

### Example 2. Modification of Phytoglycogen by its Reaction with Octenyl-Succinic Anhydride in Water

100.0 g of phytoglycogen produced according to Example 1 was dispersed in 750 mL of de-ionized water in a 2 L glass reaction vessel. The dispersion was constantly stirred and kept at 35° C. 50 mL of octenyl succinic anhydride (OSA, Sigma-Aldrich) was heated to 40° C. and was pumped into the reaction vessel. The pH was kept constant at 8.5 by adding a 4% NaOH solution to the reaction mix using an automated control system. After 100 min, the OSA pumping was stopped, and the reaction was allowed to proceed for an additional 2.5 h. Then the pH of the mixture was adjusted to 7.0 with 1 M HCl and was mixed with 3 volumes of 95% ethanol and centrifuged at 8,500×g for 15 min at 4° C. The pellet was re-suspended in water, the pH was adjusted to 7.0, and the solution was precipitated and centrifuged using the same conditions twice. Finally, the pellet containing OSA-modified phytoglycogen was dried in an oven at 50° C. for 24 h and then milled to 45 mesh. The degree of substitution determined by NMR spectroscopy was 0.27.

### Example 3. Phytoglycogen as a Non-Thickening Rheology-Modifying Ingredient that Imparts Thixotropy (Oscillatory Sweep Test)

Aqueous dispersions of 0.7% (w/w) konjac gum and 0.7% (w/w) konjac gum plus 0.7% concentration (w/w) phytoglycogen prepared according to Example 1 were used for the rheology tests.

Konjac gum is used by the food industry as a gelling agent, thickener, stabilizer, emulsifier and film former. Chemically it is a high molecular weight polysaccharide consisting primarily of mannose and glucose sugars (Glucomannan). The gum is also used in cosmetic formulations either alone or in combination with other natural gums.

The measurements were performed on a RA 2000 Rheometer (TA Instruments-Waters LLC), using a cone-plate geometry (4 cm dia plate, 1.58° steel cone; truncation gap=50.8 μm). The tests were run at 20° C. After loading the samples, there was a 4 min equilibration time, and then the stress sweep was performed at 1 Hz from 1 to 10,000 μNm (torque range). Data were collected in log mode (10 points per decade). In runs in which pre-shear was applied before the running of the stress sweeps, the compositions were subjected to a 10 Hz pre-shear for 6 min.

It was found that the viscosity of the konjac gum dispersions did not show sensitivity to pre-shear. However, pre-shearing caused drastic changes in its stress tolerance when the phytoglycogen was present: the modulus values of the samples containing phytoglycogen decreased significantly after pre-shear was applied, and the loss modulus values (G″) became larger than the storage modulus values (G′), indicating a more “liquid-like” behaviour. Without the pre-shear, G′ was greater than G″.

A formulation with this behaviour becomes “liquid-like” when mixed or shaken, and returns to its higher viscosity state after a period of time.

### Example 4. Phytoglycogen as a Non-Thickening Rheology-Modifying Ingredient that Imparts Thixotropy (Peak-Hold Test)

The test compositions, used in these experiments were 0.5% (w/w) konjac gum, either without phytoglycogen or with an additional 0.5% (w/w) phytoglycogen prepared according to Example 1.

The measurements were performed using a RA 2000 Rheometer (TA Instruments-Waters LLC), using a cone-plate geometry (4 dia cm, 1.58° steel cone; truncation gap=50.8 μm). The tests were performed at 20° C. After loading the samples, there was a 4 min equilibration time that was followed by a peak hold flow test using a 10 Hz shear rate for 1 h (sampling delay time of 10 s). After the peak hold test, a time sweep was conducted for 20 min at 1 Hz, using a 4 Pa oscillatory stress as a control variable (sampling delay time of 10 s).

Formulations without phytoglycogen did not demonstrate thixotropic behaviour (see FIG. 4).

When phytoglycogen was present, a time dependent decrease in viscosity, corresponding to thixotropic behaviour, was observed (see FIG. 4), with the viscosity decreasing rapidly during the first 2-3 min of applied shear and reaching a steady state value after ˜12 min. When shearing was stopped, the viscosity of the “undisturbed state” re-established in a short period of time (˜2 min).

These data show that the phytoglycogen nanoparticle component can impart desirable thixotropic properties to personal care formulations containing thickeners, e.g. gums.

### Example 5. Phytoglycogen as a Non-Thickening Rheology Modifying Ingredient that Imparts Thixotropy (Step Flow Loops)

The compositions used in these tests contained 0.5% (w/w) konjac gum either without phytoglycogen or with an additional 0.5% phytoglycogen prepared according to Example 1. Stepped flow loops were conducted with increasing shear rates (up flow) in the first part of the cycle, followed by decreasing shear rates (down flow) in the second part of the cycle.

The measurements were performed on a RA 2000 Rheometer (TA Instruments-Waters LLC), using a plate-cone geometry (4 dia cm, 1.58° steel cone; truncation gap=50.8 μm). The tests were performed at 20° C. After loading the samples there was a 4 min equilibration time that was followed by the stepped flow loop. In the first part of the cycle, the torque range was increased from 1 to 600 μNm. In the second part of the cycle, the torque range was decreased from 600 to 1 μNm (10 points per decade-log mode; constant time of 10 s; average over last 5 s).

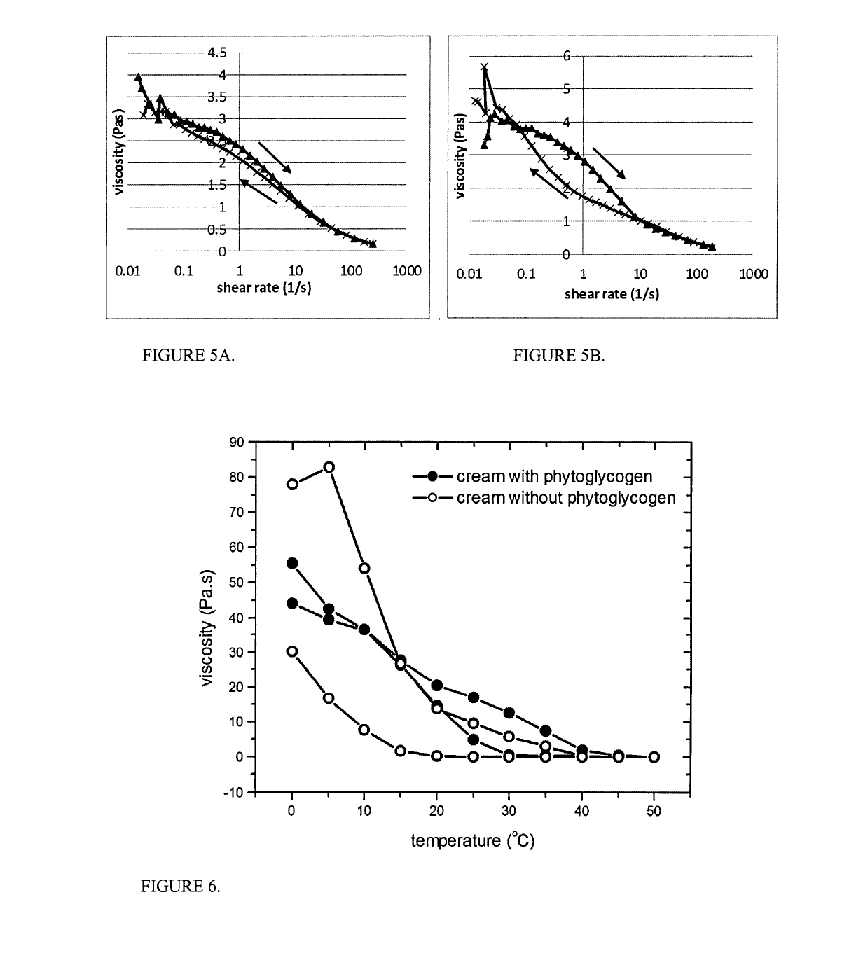

Results of the measurements are presented in FIGS. 5A and 5B. The introduction of phytoglycogen to konjac gum in the aqueous solution resulted in a significant hysteresis loop between the up and down flow curves (FIG. 5B), compared to the results obtained for phytoglycogen-free solutions (FIG. 5A). The difference between the viscosity values measured for up flow and down flow of the formulations indicates that, after shear thinning is observed with increasing flow rate, the phytoglycogen component can increase the rebuilding time and render the system thixotropic.

### Example 6. Phytoglycogen as a Rheology Stabilization Ingredient for Emulsion-Based Formulations that Provides Temperature Stress Tolerance

The effect of phytoglycogen on the rheological properties of cosmetic formulations was investigated using a commercially available “Balanced cream base” from MakingCosmetics Inc., Renton Wash., USA.

Ingredients (from the manufacturer): water, isopropyl palmitate, jojoba oil, caprylic capric triglyceride, squalane, 1,3 propanediol, ceteareth-20, dimethicone, glyceryl stearate, raspberry seed oil, cetearyl alcohol, peg-100 stearate, sodium lauryl lactylate, octyl dodecanol, beeswax, ethylhexylglycerin, caprylyl glycol, tocopheryl acetate, hydroxyethyl cellulose, hexylene glycol, disodium EDTA, tocopherol, ascorbyl palmitate, ascorbic acid, citric acid, methylisothiazolinone.

The manufacturer intends that this formulation will be customized by adding up to 15-20% (volume percentage) of additional liquid ingredients, such as active ingredients and/or fragrances, without excessive thinning of the cream.

Sample Preparation:

Formulation 1:

The cream base was combined with Milli-Q water (resistivity of 18.2 MO-cm) in the ratio of 9:1 (w/w).

Formulation 2:

The cream base was combined with a solution of 22% (w/w) phytoglycogen (prepared according to Example 1) in Milli-Q water in the ratio of 9:1 (w/w; final phytoglycogen concentration in the cream is 2.2%).

Formulation 3:

The cream base was combined with a solution of 22% OSA-modified phytoglycogen (prepared according to Example 2) in Milli-Q water in the ratio of 9:1 (w/w; final OSA-modified phytoglycogen concentration in the cream is 2.2%).

“Temperature cycling” measurements were performed using a RA 2000 Rheometer (TA Instruments-Waters LLC), using a cone-plate geometry (4 dia cm, 1.58° steel cone; truncation gap=50.8 μm). The samples were loaded onto the cold geometry and, after a 3 min equilibration time once the temperature reached 0° C., a 5 min pre-shear was performed (10 Hz). The temperature was first increased from 0° C. to 50° C., with 5° C. increments (heating cycle), and a 3 min equilibration time after each increment. The temperature was then decreased from 50° C. to 0° C. (cooling cycle) in the same manner (5° C. increments, with a 3 min equilibration time). The tests were run at 1 Hz. Two values of the torque were used: 200 μNm (“high-torque”) and 20 μNm (“low-torque”). Fresh samples were used for each “high-torque” and “low-torque” measurement.

It is common to see viscosity hysteresis between the heating and cooling phases during temperature cycling tests. The lower the hysteresis, the better the tolerance of the emulsion to the heating stress. It is also important that the viscosity recovers its original value after the heating and cooling cycles. If the viscosity value does not recover, this is an indication that an undesirable change has occurred. Such a “temperature cycling” test is designed to reproduce real life conditions, in which an emulsion-based product undergoes significant temperature variations during transportation between, for example, the manufacturing site, the warehouse and the retail store.

As can be seen from the data presented in FIG. 6 for the “high-torque” experiment, the hysteresis loop area is markedly smaller with phytoglycogen present in the formulation. Moreover, the viscosity recovered its initial value at the end of the heating and cooling cycle for Formulation 2 (containing phytoglycogen). In contrast, the viscosity of Formulation 1 without phytoglycogen was significantly lower than the viscosity at the start of the temperature cycle, which is possibly due to partial demulsification of Formulation 1. This indicated that phytoglycogen improved the emulsion stability with temperature cycling.

Formulations 1, 2 and 3 were subjected to the “low-torque” test, and measurements of the storage (G′) and loss (G″) modulus values were measured. For Formulation 1, which does not contain phytoglycogen, a large hysteresis between the heating and cooling cycles was observed for both G′ and G″ modulus values. Modulus values at the end of the temperature cycling were significantly lower than at the start, indicating possible undesirable demulsification. A significant hysteresis was also observed for Formulation 3 (containing OSA-modified phytoglycogen), since modulus values at the end the temperature cycling were larger than at the start but its presence prevented the cross over between the moduli values. Tests of Formulation 2 (containing phytoglycogen) showed the lowest hysteresis, with modulus values recovering their initial values after the experiment, and the loss modulus curve did not cross the storage modulus curve.

This result indicates that a monodisperse phytoglycogen component can improve the stability of emulsion-based formulations during temperature cycling.

### Example 7. Phytoglycogen as a Rheology Stabilization Ingredient for Emulsion-Based Formulations that Provides Tolerance to Mechanical Stress (Stress and Strain Tolerance)

The samples were prepared as in example 6.

The measurements were performed using a RA 2000 Rheometer (TA Instruments-Waters LLC), using a cone-plate geometry (4 dia cm, 1.58° steel cone; truncation gap=50.8 μm). The tests were performed at 20° C. After loading the samples, equilibration was allowed to occur for 5 min, and then the stress sweep was performed at 1 Hz using a torque range of 1 to 10,000 μNm. Data were collected in log mode (10 points per decade).

The presence of phytoglycogen or OSA-modified phytoglycogen increased the stability of the cream, as the linear viscoelastic region of these formulations extended to larger oscillatory stress or strain values when phytoglycogen or OSA-modified phytoglycogen was present.

This result indicates that a both unmodified and OSA-modified phytoglycogen can provide greater stability with respect to changes in strain and stress.

### Example 8. Phytoglycogen as a Rheology Stabilization Ingredient for Emulsion-Based Formulations that Provides Tolerance to Mechanical Stress

Formulations were prepared according to Example 6, but using two different mixing methods. The formulations were mixed either by using low shear mechanical stirring or a high-energy homogenizer (IKA T18 Basic Ultra Turrax). Use of a homogenizer is more desirable in the preparation of various emulsion-based products because it allows fast mixing and a corresponding reduction of the preparation time.

Oscillatory stress tests were used to assess the possible effect of phytoglycogen on the formulation when mixing methods with substantially different energy inputs and times necessary to complete the process were used for further customizing the base cream.

The measurements were performed using a RA 2000 Rheometer (TA Instruments-Waters LLC), using a cone-plate geometry (4 dia cm, 1.58° steel cone; truncation gap=50.8 μm). The tests were performed at 20° C. After loading the samples, equilibration was allowed to occur for 5 min, and then the stress sweep was performed at 1 Hz using a torque range of 1 to 10,000 μNm. Data were collected in log mode (10 points per decade).

Preparation of Formulation 1 (without phytoglycogen) using the high-energy homogenizer resulted in an undesirable 10-fold reduction in the viscosity of the formulation compared with that of Formulation 1 prepared using low shear mechanical stirring (3 Pa*s versus 30 Pa*s). By incorporating phytoglycogen (prepared according to Example 1) into the formulation (Formulation 2), the viscosity value obtained using the homogenizer was much closer to that obtained using low shear mechanical stirring (20 Pa*s versus 27 Pa*s). This result demonstrated that incorporation of phytoglycogen dramatically improved the stability of the emulsion to mechanical stresses. Incorporation of OSA-modified phytoglycogen (prepared according to Example 2) (Formulation 3) also resulted in reduced drop in the viscosity introduced by high-energy mixing using the homogenizer (3.4 Pa*s for the homogenizer versus 11 Pa*s for low shear mechanical stirring), but the effect was not as dramatic as for unmodified phytoglycogen (Formulation 2).

In control experiments (no phytoglycogen nanoparticles), the use of a high-shear homogenizer resulted in undesirable decreases in viscosity of the final emulsion. In contrast, simple mechanical stirring did not produce such drastic decreases in viscosity, but required an undesirably long time to mix the phases. However, incorporating monodisperse phytoglycogen additives into the formulation allowed the use of high energy mixing techniques.

This experiment demonstrates that the phytoglycogen nanoparticle component can improve the tolerance of emulsions to mechanical stresses and allow faster, high-energy mixing techniques, e.g. homogenizers, to be used.

### Example 9. Phytoglycogen Improves Sun-Protection Properties of Organic Sunscreen Formulations

Phytoglycogen and phytoglycogen modified with octenylsuccinic acid (OSA-modified phytoglycogen) were incorporated into sunscreen formulations containing homosalate (a UV-absorbing compound) as described below.

TABLE I
Sunscreen Formulation.

| | | % wt\|wt | | |
|---|---|---|---|---|
| Phase | Ingredient | I | II | III |
| Oil | Lanolin | 4.25 | 4.25 | 4.25 |
| | Petrolatum | 2.6 | 2.6 | 2.6 |
| | Stearic acid | 3.5 | 3.5 | 3.5 |
| | Stearyl palmitate | 1.7 | 1.7 | 1.7 |
| | Behenyl alcohol | 0.9 | 0.9 | 0.9 |
| | Propyl Parahydroxybenzoate | 0.05 | 0.05 | 0.05 |
| | Homosalate | 7.0 | 7.0 | 7.0 |
| Water | Deionized water | 73.65 | 67.65 | 67.65 |
| | Propylene glycol | 5.0 | 5.0 | 5.0 |
| | Phytoglycogen | | 6.0 | |
| | OSA-modified glycogen | | | 6.0 |
| | Xanthan gum | 0.2 | 0.2 | 0.2 |
| | Triethanolamine | 1.0 | 1.0 | 1.0 |
| | EDTA | 0.05 | 0.05 | 0.05 |
| | Methyl Parahydroxybenzoate | 0.1 | 0.1 | 0.1 |

Phytoglycogen was extracted from sweet corn as described in Example 1. OSA-modified phytoglycogen was prepared as described in Example 2 and the resulting degree of substitution was 0.27.

Both phases (the water and oil phases) were heated to 83° C. with constant stirring until completely melted/solubilised. The water phase was stirred with the high-energy homogenizer (IKA T18 Basic Ultra Turrax) at 24 Krpm for 2 min before mixing with the oil phase. The oil phase was then added into the water phase while stirring with the homogenizer at 24 Krpm for 2 min, cooled to 40° C. while slowly stirring, then stirred again with the homogenizer at 24 Krpm for 1 min.

The resulting sunscreen formulations were tested for the SPF value and photostability using an Optometrics SPF-290S spectrophotometer and an Ocean Optics UV-VIS spectrometer. The results of the tests are shown in Table II. The SPF boost values refer to the percentage increase in the SPF value relative to the formulation that did not contain phytoglycogen or OSA-modified phytoglycogen.

TABLE II

| | | | Crit | SPF |
|---|---|---|---|---|
| | Sunscreen actives | SPF | wavelength, nm | boost, % |
| I | Homosalate | 2.09 | 328 | 0 |
| II | Homosalate + phytoglycogen | 2.22 | 329 | 6.2 |
| III | Homosalate + OSA-modified | 2.40 | 328 | 14.8 |
| | phytoglycogen | | | |

### Example 10. Phytoglycogen Improves the Sun-Protection Properties of Inorganic Sunscreen Formulations

Phytoglycogen and OSA-modified phytoglycogen were incorporated into sunscreen formulations containing titanium dioxide using a similar procedure to that described in Example 9. The contents of the formulations are described in Table III.

TABLE III

| | | % wt/wt | | |
|---|---|---|---|---|
| Phase | Ingredient | I | II | III |
| Oil | Lanolin | 4.25 | 4.25 | 4.25 |
| | Petrolatum | 2.6 | 2.6 | 2.6 |
| | Stearic acid | 3.5 | 3.5 | 3.5 |
| | Stearyl palmitate | 1.7 | 1.7 | 1.7 |
| | Behenyl alcohol | 0.9 | 0.9 | 0.9 |
| | Propyl Parahydroxybenzoate | 0.05 | 0.05 | 0.05 |
| | Mineral oil | 7.0 | 7.0 | 7.0 |
| Water | Deionized water | 67.65 | 61.65 | 61.65 |
| | Propylene glycol | 5.0 | 5.0 | 5.0 |
| | Titanium dioxide | 6.0 | 6.0 | 6.0 |
| | Glycogen | | 6.0 | |
| | OSA-modified phytoglycogen | | | 6.0 |
| | Xanthan gum | 0.2 | 0.2 | 0.2 |
| | Triethanolamine | 1.0 | 1.0 | 1.0 |
| | EDTA | 0.05 | 0.05 | 0.05 |
| | Methyl Parahydroxybenzoate | 0.1 | 0.1 | 0.1 |

The resulting sunscreen formulations were tested for SPF and photostability using an Optometrics SPF-290S spectrophotometer and an Ocean Optics UV-VIS spectrometer. The results of the tests are shown in Table IV. The SPF boost values refer to the percentage increase in the SPF value relative to the formulation that did not contain phytoglycogen or OSA-modified phytoglycogen.

TABLE IV

| | | | Crit | SPF |
|---|---|---|---|---|
| | Sunscreen actives | SPF | wavelength, nm | boost, % |
| I | TiO₂ | 2.37 | 388 | 0 |
| II | TiO₂ + phytoglycogen | 2.64 | 388 | 11.4 |
| III | TiO₂ + OSA-modified | 2.78 | 388 | 17.3 |
| | phytoglycogen | | | |

### Example 11. Phytoglycogen Improves the Photostability of Organic Sunscreens

Phytoglycogen and OSA-modified phytoglycogen were incorporated into formulations containing chemical sunscreen actives as described in Example 9.

The formulations were deposited as thin films (surface coverage of 2-4 mg/cm²) onto a quartz plate and dried in air for 30 min. After drying, the samples were irradiated with UV light (two UV lamps, 15 W, 254 nm, UVP Inc., part #34-000-801) for 4 h and then tested for their photostability by recording optical absorption spectra.

The photodegradation was calculated from the decrease in the maximum absorption of the respective products. Also, the change in the SPF value with time of irradiation was measured for the formulations as described in Examples 9 and 10. The results are shown in Tables V, VI, VII and VIII.

TABLE V
Homosalate photostability in formulations, without or
with phytoglycogen

| | Abs @ | Abs @ 308 nm after 4 h | Change in |
|---|---|---|---|
| Sunscreen actives | 308 nm | of exposure | Abs, % |
| Homosalate 7% | 1.0 | 0.86 | −24 |
| Homosalate 7% + | 1.0 | 1.0 | 0 |
| phytoglycogen, 6% | | | |
| Homosalate 7% | 1.0 | 0.82 | −18 |
| OSA-modified | | | |
| phytoglycogen, 6% | | | |

TABLE VI
Octyl methoxycinnamate photostability in formulations,
without or with phytoglycogen

| | | Abs @ after 4 h of | Change in |
|---|---|---|---|
| Sunscreen actives | Abs @ 316 nm | exposure | Abs, % |
| OM cinnamate 7% | 1.0 | 0.26 | −74.5 |
| OM cinnamate 7% + | 1.0 | 1.30 | +30.0 |
| phytoglycogen, 6% | | | |
| OM cinnamate 7% + | 1.0 | 0.92 | −8.0 |
| OSA-modified | | | |
| phytoglycogen, 6% | | | |

TABLE VII
Avobenzone photostability in formulations, without or
with phytoglycogen.

| | | Abs @ 308 nm | |
|---|---|---|---|
| | Abs @ | after 4 h of | Change in |
| Sunscreen actives | 308 nm | exposure | Abs, % |
| Avobenzone 7% | 1.0 | 0.46 | −54.0 |
| Avobenzone 7% + phytoglycogen, | 1.0 | 1.08 | +8.0 |
| 6% | | | |
| Avobenzone 7% OSA-modified | 1.0 | 0.53 | −47.0 |
| phytoglycogen, 6% | | | |

TABLE VIII
SPF boost in irradiated sunscreen compositions containing
phytoglycogen and OSA-modified phytoglycogen.

| | | SPF after | |
|---|---|---|---|
| | | 4 h of | SPF |
| Sunscreen actives | SPF | exposure | boost, % |
| Homosalate 7% | 2.00 | 2.25 | 12.5 |
| Homosalate 7% + phytoglycogen, 6% | 2.45 | 4.2 | 71.2 |
| Homosalate 7% OSA-modified | 3.0 | 4.5 | 50.0 |
| phytoglycogen, 6% | | | |

These results show that the SPF value of the irradiated formulations was considerably higher using phytoglycogen or OSA-modified phytoglycogen. The choice of using an unmodified or OSA-modified phytoglycogen nanoparticle component will depend on the particular organic sunscreen compound.

### Example 12. Photostabilization of Vitamin A by OSA-Modified Phytoglycogen

Vitamin A and OSA-modified phytoglycogen (prepared according to Example 2) were incorporated into alcohol-based emulsions. 24 mL of water or a 20% solution of OSA-modified phytoglycogen in water was added to 74 mL of 95% ethanol under constant stirring. Then 2 g of vitamin A (retinyl acetate) was added and the mixture was stirred with a high-energy homogenizer (IKA T18 Basic Ultra Turrax) at 24 Krpm for 4 min at room temperature. This produced a stable emulsion with low viscosity, which is suitable for spray application.

Measurements were conducted as described in Example 11. Photodegradation was calculated from the decrease in the maximum absorption at 371 nm for vitamin A and at 335 nm for vitamin A-OSA-modified phytoglycogen.

The results are shown in Table IX and they demonstrate that OSA-modified phytoglycogen dramatically improved the photostability of vitamin A. After 3 h of UV light irradiation, there was no measurable change in vitamin A concentration in the emulsion containing OSA-modified phytoglycogen, in contrast to a 56% decrease in vitamin A concentration in the emulsion that did not contain OSA-modified phytoglycogen.

TABLE IX
Vitamin A photostability in formulations, without or
with OSA-modified phytoglycogen.

| | | Abs after 3 h of | Change |
|---|---|---|---|
| Actives | Abs | exposure | in Abs, % |
| Vitamin A, 2% | 1.0 | 0.44 | −56.0 |
| Vitamin A, 2% + OSA-modified | 1.0 | 1.0 | 0 |
| phytoglycogen, 6% | | | |

### Example 13. Method of Phytoglycogen Derivatization with Aminocinnamate

1 g succinoylated-phytoglycogen (DS=0.128) was dissolved in 12 ml of milliQ water and the pH was adjusted to 5. 150 mg ethyl 4-aminocinnamate was dissolved in 3 ml DMSO and mixed with the succinoylated-phytoglycogen solution. The mixture was cooled to 0° C. and 0.2 g EDAC (N-(3-Dimethylaminopropyl)-N′-ethylcarbodiimide hydrochloride), dissolved in milliQ water, was added to it with vigorous stirring. The reaction was allowed to proceed at RT for 24 h, and then the sample was precipitated with one volume of ethanol. The precipitate was re-suspended in water and the precipitation step was repeated two more times. After the last precipitation step the sample was taken up in water and was lyophilized.

The succinoylated-phytoglycogen was prepared as follows:

4 g phytoglycogen was dissolved in 34 ml DMSO and 0.3 g DMAP (4-Dimethylaminopyridine) and 0.4 g Succinic anhydride were given to the solution. The sample was incubated (with stirring) 0/N at 50° C. After the incubation 50 ml of distilled water was added to the reaction mixture and the pH was adjusted to 7. The sample was precipitated with 1 volume of ethanol. After centrifugation the pellet was dispersed in dist. water (75 ml) the pH was adjusted to 7.0-7.2. The precipitation step was repeated twice and the sample was air dried 0/N on RT then at 60° C. for two days.

### Example 14. Photostability of Phytoglycogen-Ethyl-4-Aminocinnamate Conjugate Versus Aminocinnamate

Aminocinnamate and a phytoglycogen-ethyl-4-aminocinnamate conjugate were dissolved in ethanol or in water, respectively.

Solutions were deposited as thin films onto a quartz plate and dried in air for 30 min. After drying, the samples were irradiated with UV light, as in Example 11, for 4 h and then tested for their photostability.

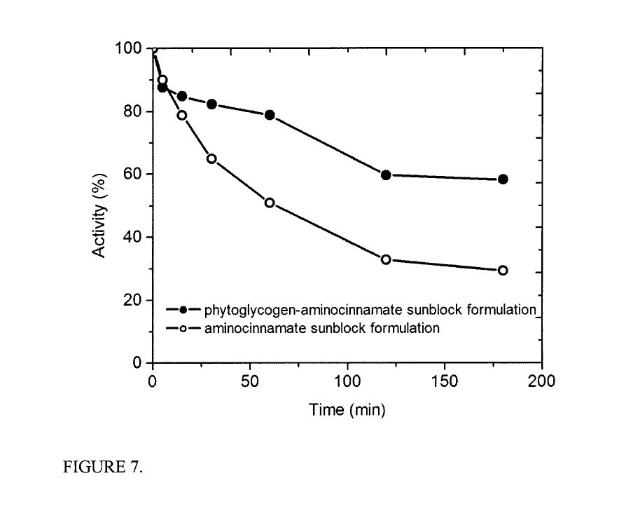

Photodegradation was calculated from the decrease in the maximum absorption of the respective products. The results are shown in FIG. 7.

It can be seen from the data shown in FIG. 7 that the phytoglycogen-ethyl-4-aminocinnamate conjugate retained 95% of its activity after 4 h of exposure to the UV-irradiation, compared to 40% of its activity for the aminocinnamate.

### Example 15. Relative Water Retention Versus Time was Compared for Phytoglycogen Nanoparticles Prepared According to Example 1, Glycerin, PEG 400 and Hyaluronic Acid

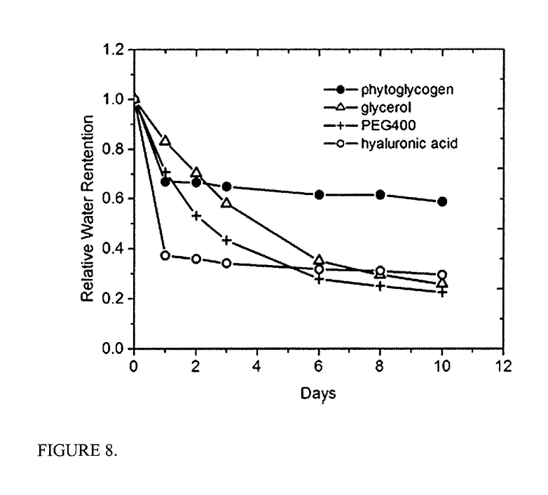

Samples were allowed to absorb water in a moisture chamber for ten days at 37° C. After quantifying the water uptake, the samples were placed in a closed chamber with desiccant and the time dependence on their relative water content was measured for ten days. The results are shown in FIG. 8, with each data point representing the average of three independent measurements.

### Example 16. Tolerance of Phytoglycogen Viscosity to Ionic Strength

Aqueous dispersions of 20% (w/w) phytoglycogen (prepared according to Example 1) were used for rheology tests.

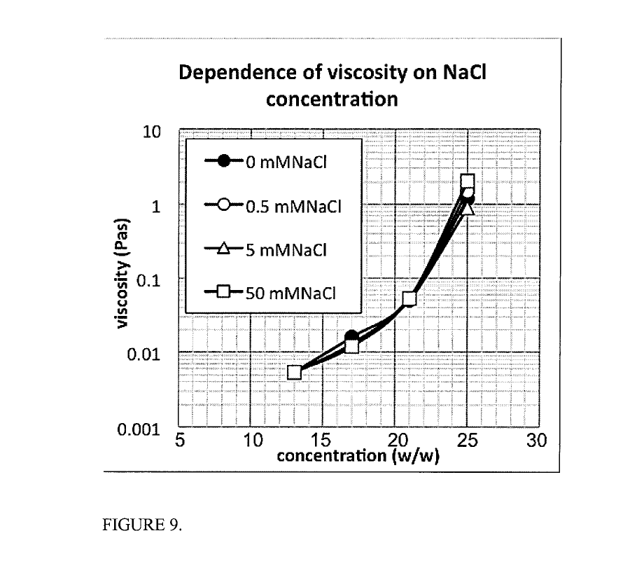

The measurements were performed on a RA 2000 Rheometer (TA Instruments-Waters LLC), using a cone-plate geometry (4 cm dia plate, 1.58° steel cone; truncation gap=50.8 μm). The tests were run at 20° C. Results are shown in FIG. 9.

### Example 17. Hydrating Effect of Combination of Monodisperse Phytoglycogen Nanoparticles and Hyaluronic Acid

The hydrating effect of untreated skin (blank), a base moisturizer with the addition of 0.1% w/w monodisperse phytoglycogen nanoparticles, 0.1% w/w hyaluronic acid, and with both monodisperse phytoglycogen nanoparticles (0.05% w/w) and hyaluronic acid (0.05% w/w) were compared.

Formulations for hydration tests are shown in Table X below.

| | | | | | 0.05% |
|---|---|---|---|---|---|
| | | | | 0.1% | Phytoglycogen + |
| | | | 0.1% | Hyaluronic | 0.05% Hyaluronic |
| Phase | Ingredient | Base | Phytoglycogen | acid | acid |
| A | Water | 73.1 | 73.0 | 73.0 | 73.0 |
| | Glycerin | 5.0 | 5.0 | 5.0 | 5.0 |
| | Xanthan gum | 0.5 | 0.5 | 0.5 | 0.5 |
| | Phytoglycogen | | 0.1 | | 0.05 |
| | Hyaluronic acid | | | 0.1 | 0.05 |
| B | Almond oil | 5.0 | 5.0 | 5.0 | 5.0 |
| | Avocado oil | 5.0 | 5.0 | 5.0 | 5.0 |
| | Shea butter | 4.0 | 4.0 | 4.0 | 4.0 |
| | Xiameter PMX-200, | 2.0 | 2.0 | 2.0 | 2.0 |
| | 100 CS | | | | |
| | (Dimethicone) | | | | |
| | Cetyl alcohol | 2.0 | 2.0 | 2.0 | 2.0 |
| | Stearyl alcohol | 1.0 | 1.0 | 1.0 | 1.0 |
| | Sorbitan stearate | 1.4 | 1.4 | 1.4 | 1.4 |
| C | Tocopherol acetate | 0.2 | 0.2 | 0.2 | 0.2 |
| | (vitamin E) | | | | |
| | Phenoxyethanol, | 0.8 | 0.8 | 0.8 | 0.8 |
| | sorbic acid, caprylyl | | | | |
| | glycol | | | | |
| D | Triethanolamine | * | * | * | * |
| | Citric acid (50% w/w) | * | * | * | * |

Phases A and B were heated to 75° C. with constant stirring until completely melted/solubilised. The oil phase was then added into the water phase while stirring at 800 rpm with a Caframo Stirrer, type RZR50, equip with a circular teethed blade. Phase C was added when the temperature dropped below 40° C. and the emulsion continued to mix for 5 minutes at 500 rpm. The final pH was adjusted to 5.5-6.0 with phase D using an IQ Scientific mini lab ISFET pH meter (model IQ125).

Skin hydration was measured using the MoistureMeterSC™ by Delfin Technologies, used in accordance with manufacturer's directions. Delfin Modular Core™ (DMC) software was used to setup tests and record results.

Volunteers were asked to arrive 1 hour before testing began to acclimatize to the test environment, and to remain indoors all day. In addition, the volunteers were directed that only desk work should be done and that the test site (inner forearms) were to be kept isolated throughout the day as best as possible.

A bar of Ivory soap (Proctor & Gamble) was used to wash the inner forearms and paper towel was used to pat the skin dry. Forearms were washed at least 15 minutes prior to beginning the test to allow hydration and TEWL values to stabilize. Rectangular areas measuring 2.5×3.8 cm² were marked on the inner forearms using permanent marker.

Samples from each cream were weighed out in the lab on the day of the test. Gloves were worn to avoid contamination and the creams were stirred using a clean spatula. 0.02 g of tested products were weighed out on tared aluminum foil pieces ˜3×3 cm².

Measurements of each site were taken before samples were applied (T=0). Measurements were taken in triplicate, by the same person, and were recorded using the Delfin Modular Core (DMC) software.

Samples were then self-applied to the marked areas and were rubbed into the skin using a finger, with a different finger being used to rub in each different sample.

Subsequent measurements were taken after 1 hour, 3 hours, 5 hours, and 7 hours.

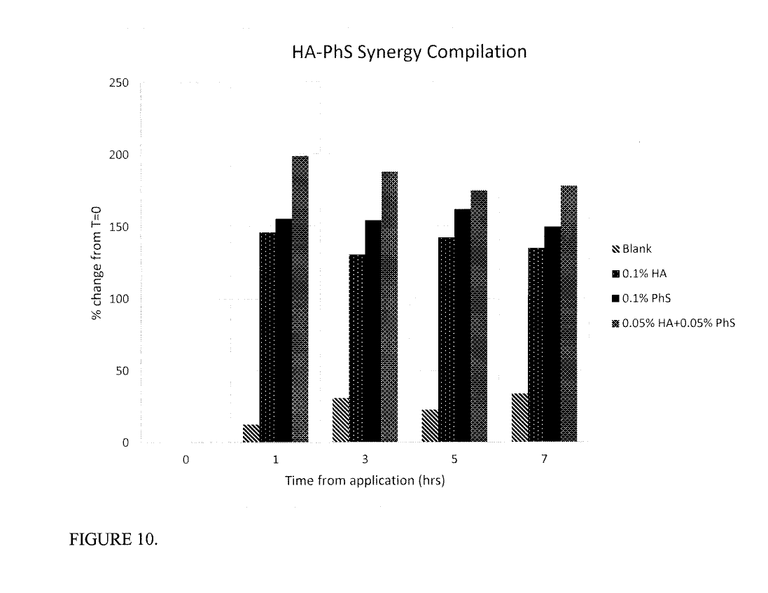

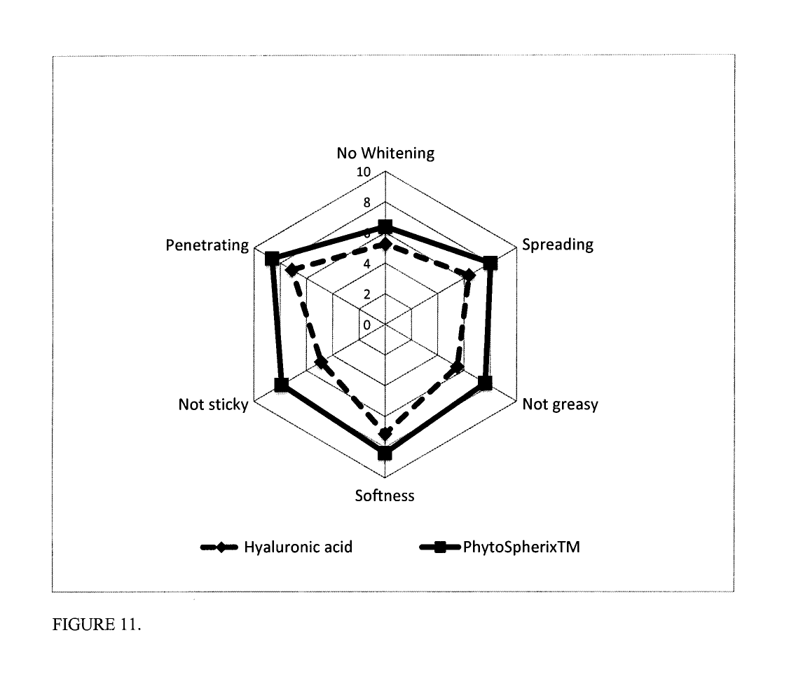

Untreated skin (blank), a base moisturizer with the addition of 0.1% w/w monodisperse phytoglycogen nanoparticles, 0.1% w/w hyaluronic acid (Making Cosmetics, MW=800-1200 Da) and with both monodisperse phytoglycogen nanoparticles (0.05% w/w) and hyaluronic acid (0.05% w/w) was tested at 1, 3, 5 and 7 hours post application. As illustrated in FIG. 10, the base moisturizer with both monodisperse phytoglycogen nanoparticles and hyaluronic acid had superior hydrating properties at all time points.

### Example 18. Sensory Analysis of Moisturizers Containing Hyaluronic Acid Alone or Monodisperse Phytoglycogen Nanoparticles Alone

Moisturizers prepared using the same base formulation and hyaluronic acid alone or the same amount of monodisperse phytoglycogen nanoparticles alone were tested by participants in a blind test and were evaluated on six features: no whitening, spreading, not greasy, soft, not sticky, and penetrating.

Formulations for sensory analysis tests are shown in Table XI below.

| | | % wt/wt | |
|---|---|---|---|
| | | 0.5% | 0.5% Hyaluronic |
| Phase | Ingredient | Phytoglycogen | acid |
| A | Water | 70.9 | 70.9 |
| | Colloidal oatmeal | 1.5 | 1.5 |
| | Glycerin | 5.0 | 5.0 |
| | Xanthan gum | 0.7 | 0.7 |
| | Phytoglycogen | 0.5 | |
| | Hyaluronic acid | | 0.5 |
| B | Almond oil | 5.0 | 5.0 |
| | Jojoba oil | 5.0 | 5.0 |
| | Shea butter | 4.0 | 4.0 |
| | Xiameter PMX-200 fluid, | 2.0 | 2.0 |
| | 100 cs (dimethicone) | | |
| | Cetyl alcohol | 2.0 | 2.0 |
| | Stearyl alcohol | 1.0 | 1.0 |
| | Sorbitan stearate | 1.4 | 1.4 |
| C | Tocopherol acetate | 0.2 | 0.2 |
| | (vitamin E) | | |
| | Phenoxyethanol, sorbic | 0.8 | 0.8 |
| | acid, caprylyl glycol | | |

The results are illustrated in FIG. 11. All participants preferred the moisturizer formulated with monodisperse phytoglycogen nanoparticles.

Based on a visual inspection, emulsions containing phytoglycogen showed improved stability after one week stored at 45° C.; specifically, the hyaluronic acid sample showed physical signs of phase separation and a slight change in colour, indicating instability.

### Example 19. Phytoglycogen Nanoparticle and Polysaccharide Films

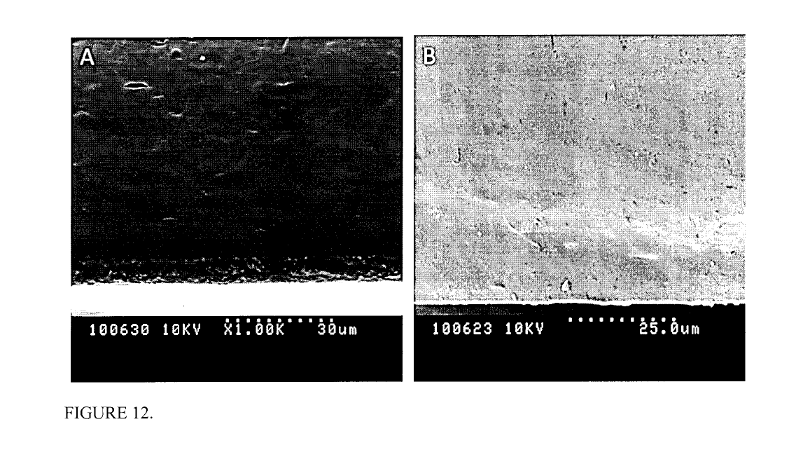

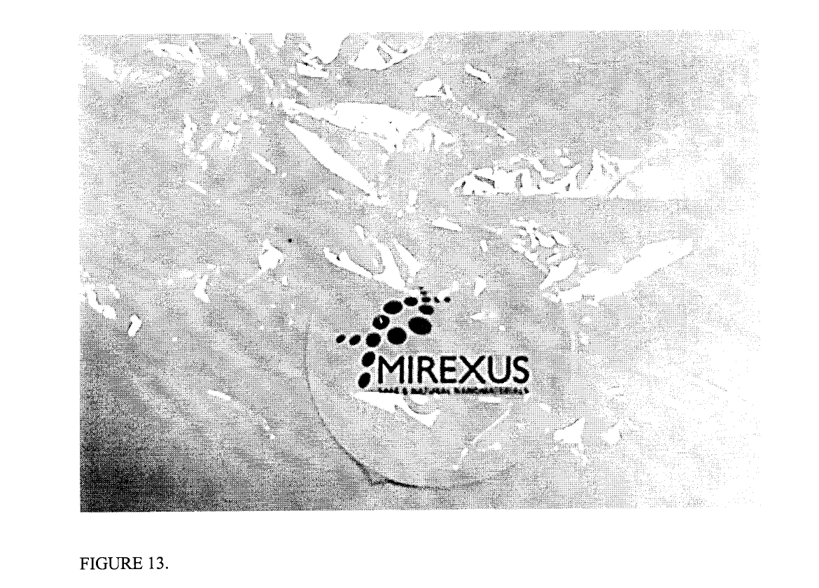

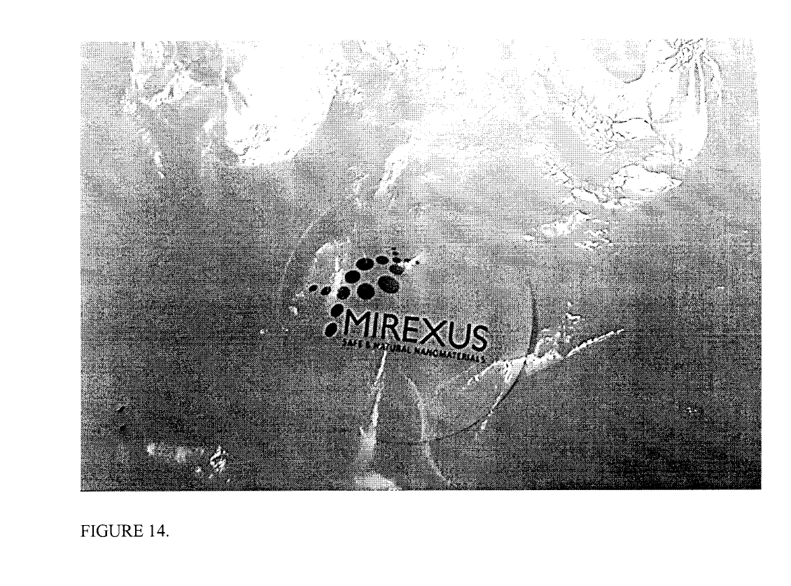

Polymer films were prepared using sodium hyaluronate or sodium alginate or chitosan polysaccharides in combination with phytoglycogen nanoparticles. All composite films were optically clear, and smooth enough to reflect light from their surfaces (see FIGS. 12, 13 and 14, indicating no phase separation or aggregation, and a homogeneous mixing of the components at the molecular level. Without being bound by a theory, this suggests a chemical synergy whereby individual phytoglycogen nanoparticles are bound to the polysaccharide component.

Sodium hyaluronate films were prepared by mixing equal parts of a 1% solution of sodium hyaluronate solution with a 1% phytoglycogen solution, then drop-casting onto a plexiglass plate and allowing to dry for 12 hours. Sodium alginate films were prepared by mixing equal parts of a 2% solution of sodium alginate solution with a 2% phytoglycogen solution, then drop-casting onto a plexiglass plate and allowing to dry for 12 hours. Chitosan-based polymer films were prepared by drop-casting weakly acidic polymer solutions into a 6″ metal cylinder placed on a plexiglass surface. Pure chitosan films (FIG. 12A) were prepared using 2% chitosan in a 2% acetic acid solution, while the composite films (FIG. 12B) were prepared using a 1% chitosan and 1% phytoglycogen nanoparticles in a 2% acetic acid solution. The solutions were dried in a dessicated environment for 48 hours and resulting films were peeled off of the plexiglass surface.

Pure phytoglycogen films and pure chitosan films were very brittle, easily breaking when bent more than ˜30 degrees. Surprisingly, the blended composite was notably tougher than either pure film, and could be bent and even creased without snapping.

Samples of each chitosan-phytoglycogen film were frozen in liquid nitrogen, broken in two, warmed back to room temperature and imaged along the cleaved edge using SEM. The images show no obvious morphological features and no indication of aggregation, indicating these two polysaccharides are very compatible and interact strongly with each other, resulting in a uniform dispersion of nano-scale phytoglycogen nanoparticles throughout the chitosan medium.

The composite polysaccharide-phytoglycogen nanoparticle materials are flexible, and made of natural carbohydrate polymers, each of which is biocompatible and holds water very strongly. This makes it useful as a basis for cosmetic applications such as face masks, dermatological applications such as patches for dry or damaged skin, or biomedical applications such as wound dressings or subdermal implants.

### Example 20. Using Phytoglycogen Nanoparticles to Modify the Rheology of Hyaluronic Acid Solutions

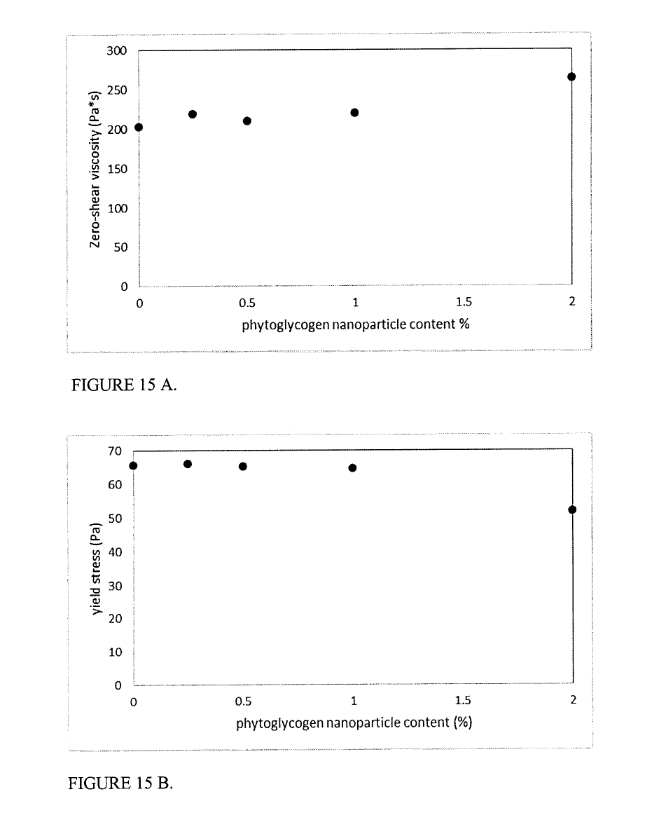

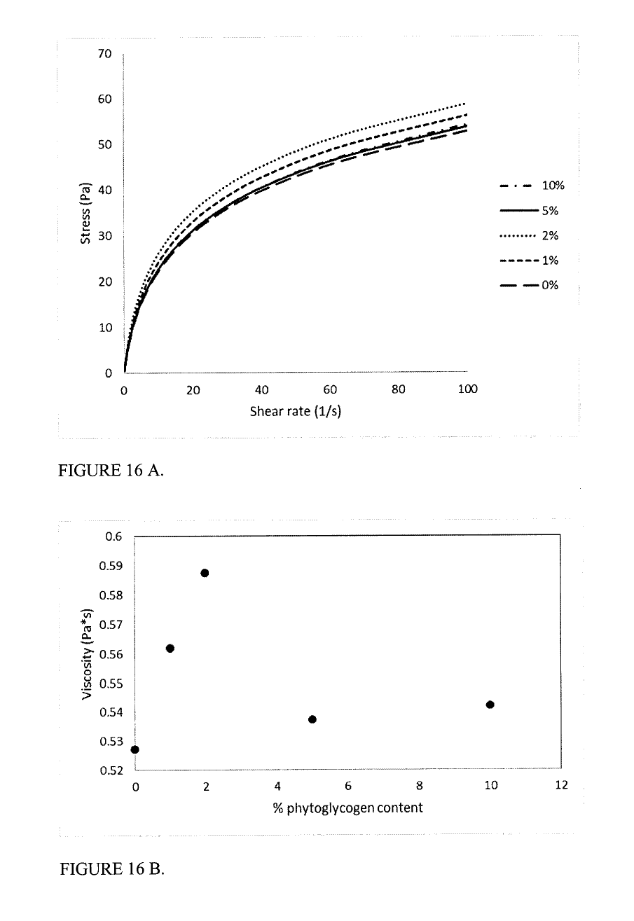

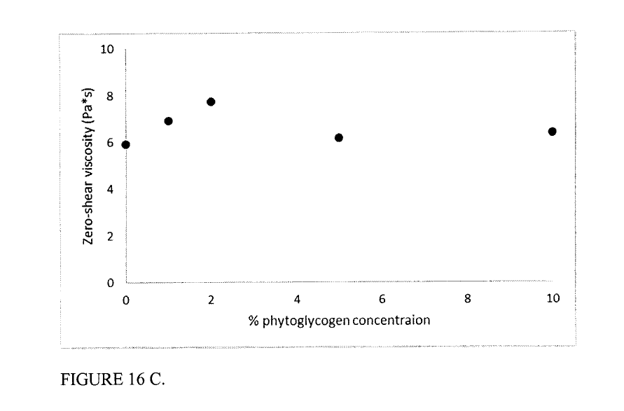

A 2.2% w/v solution of hyaluronic acid (Bloomage Freda) was prepared in 0.01 M phosphate buffered saline (PBS). Phytoglycogen nanoparticles were added to the stock solution, along with additional PBS, to produce samples with final concentrations of 0%, 0.25%, 0.5%, 1%, and 2% phytoglycogen nanoparticles. The zero-shear viscosity and yield stress where measured using a TA Instruments, Discovery Hybrid Rheometer and a 40 mm, 2° cone and plate geometry at 25° C. The zero-shear viscosity was determined by the Carreau-Yasuda model and yield stress was determined from a 5% deviation in the linear region of the storage modulus as a function of strain. These values did not significantly deviate from hyaluronic acid alone up until 2% phytoglycogen nanoparticle loading, when the yield stress decreased (results shown in FIGS. 15A and 15B). The addition of phytoglycogen nanoparticles to 1% w/w hyaluronic acid (Making Cosmetics Inc.) solutions in water at ratios of 1:0, 1:1, 1:2, 1:5 and 1:10 also improved the shear thinning properties (FIG. 16 A). A maximum effect was observed at 1:2 phytoglycogen loading (FIG. 16 B). The zero-shear viscosity for these samples was also found to not deviate significantly from hyaluronic acid alone, with a maximum deviation at 1:2 phytoglycogen loading (FIG. 16 C).

A decrease in the yield stress and an increase in shear-thinning will allow the sample to flow with less applied force. This will also enhance the sensory profile of final products and benefit the ease of spreadability and result in more uniform application for better coverage. Formulations can also be more easily processed, requiring less energy to initiate flow and mix. Additionally, concentrations up to 10% w/v phytoglycogen nanoparticles can be added to hyaluronic acid formulations without significantly impacting the viscosity, making these materials compatible and easy to incorporate into existing formulations.

### Example 21. Improved Water Retention Properties of Lyophilized Hyaluronic Acid and Phytoglycogen Powders

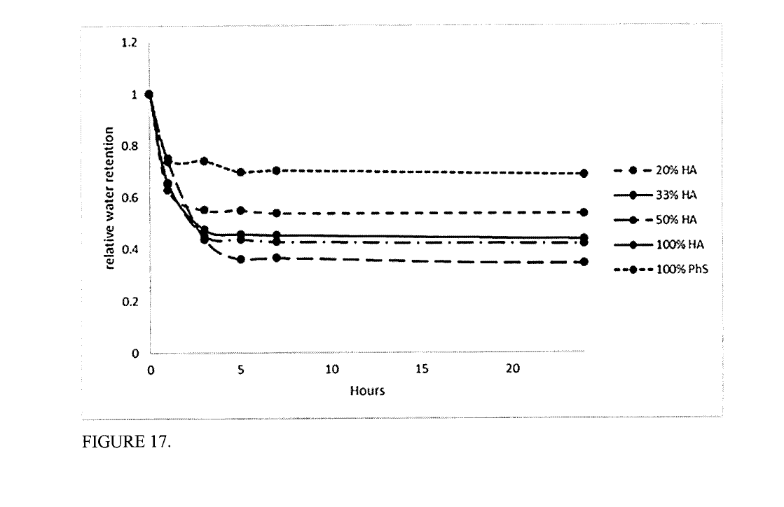

Solutions containing hyaluronic acid and phytoglycogen nanoparticles in combinations of 100% hyaluronic acid, 50% hyaluronic acid+50% phytoglycogen nanoparticles, 33% hyaluronic acid+67% phytoglycogen nanoparticles, 20% hyaluronic acid+80% phytoglycogen nanoparticles, and 100% phytoglycogen nanoparticles were lyophilized. 100 mg of each sample was placed in a desiccator until a stable dry weight was reached. The samples were then transferred to a 97% RH chamber and weighed each day to monitor water sorption activity. When the samples were fully hydrated and a steady state had been established, they were transferred back to the desiccator so that water retention could be monitored through gravimetric analysis. It was found that samples containing a higher fraction of phytoglycogen nanoparticles to hyaluronic acid retained more water than samples with a higher hyaluronic acid content. These results are summarized in FIG. 17. Being able to retain moisture for longer periods of time imparts better moisturizing properties to cosmetic formulations.

### Example 22. Sensory Analysis of Moisturizers Containing Hyaluronic Acid Alone or in Combination with Phytoglycogen Nanoparticles

Formulations of the same base with either 0.1% hyaluronic acid or 0.05% hyaluronic acid+0.05% phytoglycogen nanoparticles were prepared according to Table XII below:

| | | % wt/wt quantity | |
|---|---|---|---|
| | | | 0.05% |
| | | 0.1% | Phytoglycogen + |
| | | Hyaluronic | 0.05% Hyaluronic |
| Phase | Ingredient | acid | acid |
| A | Water | 78.0 | 78.0 |
| | Xanthan gum | 0.5 | 0.5 |
| | Phytoglycogen | — | 0.05 |
| B | Hyaluronic acid | 0.1 | 0.05 |
| | Almond oil | 5.0 | 5.0 |
| | Avocado oil | 5.0 | 5.0 |
| | Shea butter | 4.0 | 4.0 |
| | Xiameter PMX-200, | 2.0 | 2.0 |
| | 100 CS | | |
| | (Dimethicone) | | |
| | Cetyl alcohol | 2.0 | 2.0 |
| | Stearyl alcohol | 1.0 | 1.0 |
| | Sorbitan stearate | 1.4 | 1.4 |
| C | Tocopherol acetate | 0.2 | 0.2 |
| | (vitamin E) | | |
| | Phenoxyethanol, | 0.8 | 0.8 |
| | sorbic acid, caprylyl | | |
| | glycol | | |
| D | Triethanolamine | * | * |
| | Citric acid (50% w/w) | * | * |

Phases A and B were heated to 75° C. with constant stirring until completely melted/solubilised. The oil phase was then added into the water phase while stirring at 800 rpm with a Caframo Stirrer, type RZR50, equip with a circular teethed blade. Phase C was added when the temperature dropped below 40° C. and the emulsion continued to mix for 5 minutes at 500 rpm. The final pH was adjusted to 5.5-6.0 with phase D added dropwise and measured using a SympHony SB70P pH meter and Oakton Instruments 12 mm single-junction epoxy body gel-filled pH electrode.

Participants in a blind study were asked to evaluate the formulations on twelve qualities: opacity, whiteness, uniformity, thickness, ability to retain structure, ease of spreading, absorption (immediately after application and 2 min later), greasiness, softness, tackiness, irritation (immediately after application and 2 min later), and shine. This was done by ranking the qualities on a scale of 1-10 for both products. Participants were also asked to choose their preferred formulation overall.

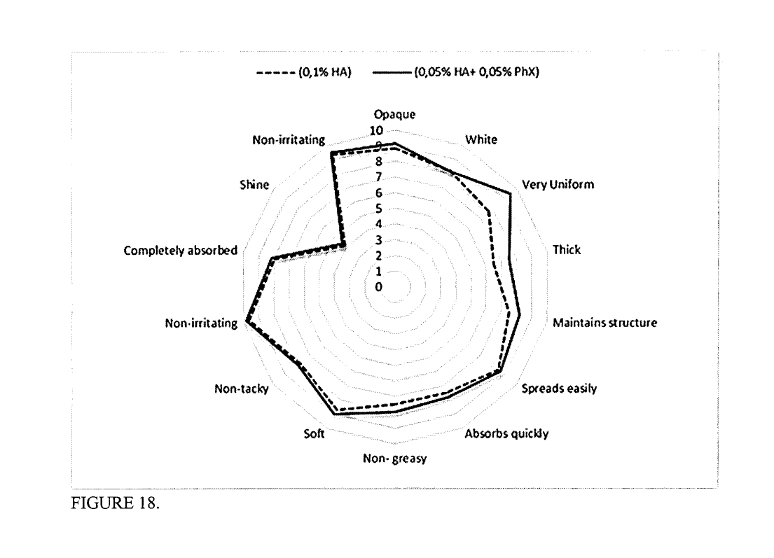

It was found that both formulations were ranked very similarly in most qualities, with differences in uniformity, thickness, and ability to maintain structure. However, 67% of participants preferred the 0.05% hyaluronic acid+0.05% phytoglycogen nanoparticles formulation overall (compared to 33% who preferred the 0.1% hyaluronic acid formulation). Results of this study are summarized in FIG. 18.

### Example 23. Stability of Emulsions Containing Hyaluronic Acid and Phytoglycogen Nanoparticles

Emulsions containing 0.1% hyaluronic acid, 0.1% phytoglycogen nanoparticles, 0.05% hyaluronic acid+0.05% phytoglycogen nanoparticles, and a base containing neither active, were prepared according to the formulation shown in Table XIII below:

| | | | 0.05% | | |
|---|---|---|---|---|---|
| | | 0.1% | Phytoglycogen + | | |
| | | Hyaluronic | 0.05% Hyaluronic | 0.1% | |
| Phase | Ingredient | acid | acid | Phytoglycogen | Base |
| A | Potassium cetyl | 1 | 1 | 1 | 1 |
| | phosphate | | | | |
| | Cetearyl alcohol | 1.5 | 1.5 | 1.5 | 1.5 |
| | Caprylic/capric | 10 | 10 | 10 | 10 |
| | triglyceride | | | | |
| | Phenoxyethanol, | 1 | 1 | 1 | 1 |
| | ethylhexylglycerin | | | | |
| B | Glycerin | 3 | 3 | 3 | 3 |
| | Aqua | 82.4 | 82.4 | 82.4 | 82.5 |
| | Sodium hyaluronate | 0.1 | 0.05 | — | — |
| | Phytoglycogen | — | 0.05 | 0.1 | — |
| C | Polyacrylamide, | 1 | 1 | 1 | 1 |
| | C13-14 isoparaffin, | | | | |
| | Laureth-7 | | | | |

Phases A and B were combined separately. A was heated to 90-95° C. and B was heated to 80-85° C. B was then added to A under constant stirring and homogenized at 13,000 rpm for 1 min. C was then added under constant stirring while cooling. At 35° C. the emulsion was homogenized for 1 min at 9,000 rpm, the pH was checked, then the emulsion was stirred for another 15 min.

To assess stability, the samples were then centrifuged for 15 min at 6000 rpm twice and evaluated for signs of phase separation. The centrifuge test is an accelerated stability test often used in the cosmetics industry to predict if a formulation will be stable. Unstable formulations will show signs of phase separation whereas stable formulations will not. The sample containing 0.1% hyaluronic acid was found to show some separation on the bottom, but all other samples appeared to be stable. Therefore, the addition of phytoglycogen nanoparticles to hyaluronic acid formulations improved emulsion stability.

1. A personal care composition comprising a monodisperse phytoglycogen nanoparticle component and a moisturizing polysaccharide component.

2. The composition of claim 1, wherein the moisturizing polysaccharide component comprises at least one of a glycosaminoglycan, a chitosan, an alginate or a beta glucan or a similar or derivative polysaccharide.

3. The composition of claim 1, wherein the moisturizing polysaccharide component comprises a glycosaminoglycan or a derivative thereof.

4. The composition of claim 1, wherein the moisturizing polysaccharide component comprises a chitosan, an alginate and/or a beta glucan.

5. The composition of claim 3, wherein the moisturizing polysaccharide component is hyaluronic acid.

6. The composition of claim 2, wherein the composition is a water-based formulation.

7. The composition of claim 2, wherein the composition is an alcohol-based formulation.

8. The composition of claim 7, wherein the alcohol is ethyl alcohol, propyl alcohol, isopropyl alcohol, ethylene glycol, propylene glycol, butylene glycol, dipropylene glycol, ethoxydiglycol, or glycerol or a combination thereof.

9. The composition of claim 2 wherein the phytoglycogen nanoparticle component has a polydispersity index of less than about 0.3, 0.2 or 0.1 as measured by dynamic light scattering.

10. (canceled)

11. The composition of claim 9, wherein ≥about 90% by dry weight of the phytoglycogen nanoparticle component is monodisperse phytoglycogen nanoparticles having an average particle diameter of between about 30 nm and about 150 nm.

12. The composition of claim 11, wherein ≥about 90% by dry weight of the phytoglycogen nanoparticle component is monodisperse phytoglycogen nanoparticles having an average particle size of between about 60 nm and about 110 nm.

13. The composition of claim 2 wherein the monodisperse phytoglycogen nanoparticles are chemically modified.

14. The composition of claim 13, wherein the monodisperse phytoglycogen nanoparticles are modified by chemical functionalization of at least one of its hydroxyl groups with a carbonyl group, an amine group, a thiol group, a carboxylic group, or a hydrocarbyl.

15. The composition of claim 14, wherein the hydrocarbyl group is an alkyl, vinyl or allyl group.

16. The composition of claim 13, wherein the monodisperse phytoglycogen nanoparticles are modified with octenyl succinic acid.

17. The composition of claim 2 wherein the phytoglycogen nanoparticle component and the moisturizing polysaccharide component are present in a concentration of up to about 25% w/w.

18. The composition of claim 17, wherein the phytoglycogen nanoparticle component and the moisturizing polysaccharide component are present in a concentration of between about 0.05 and about 5% w/w.

19. The composition of claim 17 wherein the phytoglycogen nanoparticle component and the moisturizing polysaccharide component are present in substantially equal amounts.

20. The composition of claim 17 wherein the personal care formulation is a lotion, a gel, a mask, a sunscreen, a sanitizer, a shampoo, a conditioner, a deodorant, an antiperspirant, an aftershave, a hand sanitizer, a hairspray or a cosmetic.

21. The composition of claim 17, wherein the formulation further comprises a natural gum.

22. The composition of claim 17 further comprising at least one small molecule, polymer, biopolymer, colloidal particle or an oil.

23. (canceled)

24. The composition of claim 17 further comprising a cosmetically-acceptable carrier comprising one or more ingredients selected from absorbents, anti-acne agents, anti-caking agents, anti-cellulite agents, anti-foaming agents, anti-fungal agents, anti-inflammatory agents, anti-microbial agents, antioxidants, antiperspirant/deodorant agents, anti-viral agents, anti-wrinkle agents, artificial tanning agents, astringents, binders, buffering agents, bulking agents and fillers, chelating agents, colouring agents and dyes, emollients, emulsifiers, enzymes, essential oils, exfoliating agents, film formers, flavors, foaming agents, fragrances and perfumes, humectants, hydrating agents, hydrocolloids, light diffusers, lightening agents, opacifiers, optical brighteners and modifiers, particulates, pH adjusters, preservatives, sequestering agents, skin conditioners and moisturizers, skin feel modifiers, skin protectants, skin soothing and healing agents, solvents, stabilisers, sunscreen actives, topical anesthetics, viscosity modifiers, vitamins, and combinations thereof.

25. The composition of claim 17 in the form of a powder.

26. (canceled)

27. (canceled)

28. The composition of claim 17, wherein the composition is a spray on personal care composition.

29. (canceled)
